(19) United States
(12) Patent Application Publication
(10) Pub. No.: US 2011/0297235 A1
(43) Pub. Date: Dec. 8, 2011

(54) USE OF TRIARYLAMINE DERIVATIVES AS HOLE-CONDUCTING MATERIALS IN ORGANIC SOLAR CELLS AND ORGANIC SOLAR CELLS CONTAINING SAID TRIARYLAMINE DERIVATIVES

(75) Inventor: Hermann Bergmann, Singapore (SG)

(73) Assignee: BASF SE, Ludwigshafen (DE)

(21) Appl. No.: 13/202,878

(22) PCT Filed: Feb. 15, 2010
(86) PCT No.: PCT/EP2010/051826

§ 371 Date: Aug. 23, 2011

(30) Foreign Application Priority Data
Feb. 23, 2009 (EP) 09153460.2

(51) Int. Cl. H01L 51/46; C07D 209/88; C07D 409/04; C07C 211/54

(52) U.S. Cl. 136/263; 564/434; 548/442; 549/59

(57) ABSTRACT

The present invention relates to the use of compounds of the general formula I

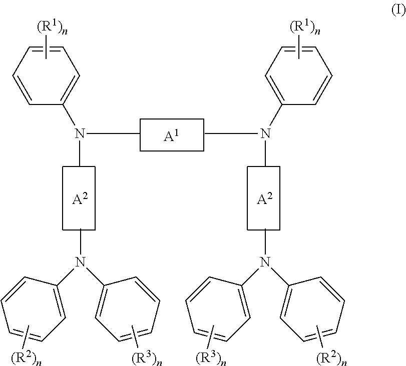

in which
A¹, A², A³ are each independently divalent organic units which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R¹, R², R³ are each independently R, OR, NR₂, A³-OR or A³-NR₂ substituents,
R is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl or NR′ radical,
R′ is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
   and
n at each instance in formula I is independently 0, 1, 2 or 3,
   with the proviso that the sum of the individual values n is at least 2 and at least two of the R¹, R² and R³ radicals are OR and/or NR₂ substituents,
      as hole-conducting materials in organic solar cells.

The present invention further relates to organic solar cells which comprise these compounds.

The present invention relates to the use of compounds of the general formula I

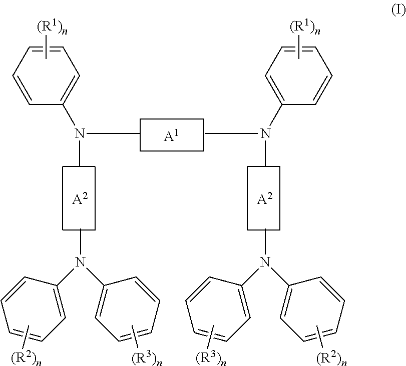

in which
A¹, A², A³ are each independently divalent organic units which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R¹, R², R³ are each independently R, OR, NR₂, A³-OR or A³-NR₂ substituents,
R is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl or NR′ radical,
R′ is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
   and
n at each instance in formula I is independently 0, 1, 2 or 3,
   with the proviso that the sum of the individual values n is at least 2 and at least two of the R¹, R² and R³ radicals are OR and/or NR₂ substituents,
      as hole-conducting materials in organic solar cells.

The present invention further relates to organic solar cells which comprise these compounds.

Dye solar cells (“DSCs”, or dye-sensitized solar cells, “DSSCs”; these terms and abbreviations are used synonymously hereinafter) are one of the most efficient alternative solar cell technologies at present. In a liquid variant of this technology, efficiencies of up to 11% have been achieved to date (e.g. Grätzel M. et al., J. Photochem. Photobio. C, 2003, 4, 145; Chiba et al., Japanese Journal of Appl. Phys., 2006, 45, L638-L640).

The construction of a DSC is generally based on a glass substrate, which is coated with a transparent conductive layer, the working electrode. An n-conductive metal oxide is generally applied to this electrode or in the vicinity thereof, for example an approx. 10-20 μm-thick nanoporous titanium dioxide layer (TiO₂). On the surface thereof, in turn, a monolayer of a light-sensitive dye, for example a ruthenium complex, is typically adsorbed, which can be converted to an excited state by light absorption. The counterelectrode may optionally have a catalytic layer of a metal, for example platinum, with a thickness of a few μm. The area between the two electrodes is filled with a redox electrolyte, for example a solution of iodine (I₂) and lithium iodide (LiI).

The function of the DSC is based on the fact that light is absorbed by the dye, and electrons are transferred from the excited dye to the n-semiconductive metal oxide semiconductor and migrate thereon to the anode, whereas the electrolyte ensures that the charges are balanced via the cathode. The n-semiconductive metal oxide, the dye and the (usually liquid) electrolyte are thus the most important constituents of the DSC, though cells comprising liquid electrolyte in many cases suffer from nonoptimal sealing, which leads to stability problems. Various materials have therefore been studied for their suitability as solid electrolytes/p-semiconductors.

For instance, various inorganic p-semiconductors such as CuI, CuBr.3(S(C₄H₉)₂) or CuSCN have found use to date in solid-stage DSCs. With CuI- or CuSCN-based, solid DSCs, for example, efficiencies of up to 3% have been reported (Tennakone et al. J. Phys. D: Appl. Phys, 1998, 31, 1492; O'Regan et al. Adv. Mater 200, 12, 1263; Kumara et al. Chem. Mater. 2002, 14, 954).

Organic polymers are also used as solid p-semiconductors. Examples thereof include polypyrrole, poly(3,4-ethylenedioxythiophene), carbazole-based polymers, polyaniline, poly(4-undecyl-2,2′-bithiophene), poly(3-octylthiophene), poly(triphenyldiamine) and poly(N-vinylcarbazole). In the case of poly(N-vinylcarbazole), the efficiencies reach up to 2%; with a PEDOT (poly(3,4-ethylenedioxythiophene), polymerized in situ, an efficiency of 2.9% was even achieved (Xia et al. J. Phys. Chem. C 2008, 112, 11569), though the polymers are typically not used in pure form but usually in a mixture with additives. In addition, a concept in which polymeric p-semiconductors are bonded directly to an Ru dye is also presented (Peter, K., Appl. Phys. A 2004, 79, 65).

The highest efficiencies to date are, however, achieved with low molecular weight organic p-semiconductors. For example, a vapor-deposited layer of triphenylamine (TPD) was applied as a replacement for the liquid electrolyte to the dye-sensitized layer. The use of the organic compound 2,2′,7,7′-tetrakis(N,N-di-p-methoxyphenyl-amine)-9,9′-spirobifluorene (spiro-MeOTAD) in dye solar cells was reported in 1998. The methoxy groups adjust the oxidation potential of the spiro-MeOTAD such that the Ru complex can be regenerated efficiently. In the case of use of spiro-MeOTAD alone as a p-conductor, a maximum IPCE (incident photon to current conversion efficiency, external photon conversion efficiency) of 5% is achieved. With addition of N(PhBr)₃SbCl⁶ (as a dopant) and Li[(CF₃SO₂)₂N], a rise in the IPCE to 33%, and in the efficiency to 0.74%, was observed. The addition of tert-butylpyridine enhanced the efficiency to 2.56%, with an open-circuit voltage (V_{oc}) of approx. 910 mV and a short-circuit current I_{SC} of approx. 5 mA measured at an active area of approx. 1.07 cm² (Krüger et al., Appl. Phys. Lett., 2001, 79, 2085). Dyes which achieve better coverage of the TiO₂ layer and which have good wetting on spiro-MeOTAD exhibit efficiencies of more than 4% (Schmidt-Mende et al., Adv. Mater. 17, p. 813-815 (2005)). Even better efficiencies of 5.1% are observed when a ruthenium complex with oxyethylene side chains is used (Snaith, H. et al., Nano Lett., 7, 3372-3376 (2007)).

Durrant et al., Adv. Func. Mater. 2006, 16, 1832-1838 state that, in many cases, the photocurrent is directly dependent on the yield in the hole transition from the oxidized dye to the solid p-conductor. This depends essentially on two factors: first on the degree of penetration of the p-semiconductor into the oxide pores, and second on the thermodynamic driving force for the charge transfer, i.e. especially on the difference in the free enthalpy ΔG between dye and p-conductor.

Currently the best efficiencies in DSCs with solid hole conductors are obtained with the compound spiro-MeOTAD already mentioned above, the chemical formula of which is shown below:

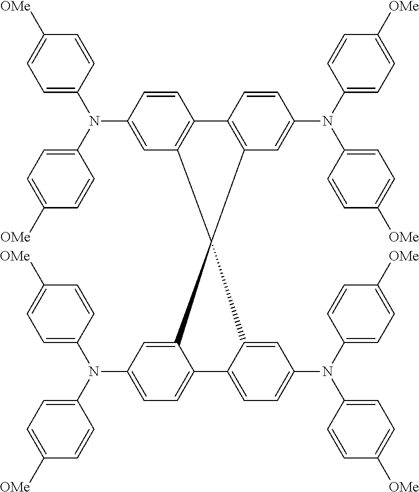

### (Snaith, H. J.; Moule, A. J.; Klein, C.; Meerholz, K.; Friend, R. H.; Gratzel, M. Nano Lett.; (Letter); 2007; 7(11); 3372-3376)

Studies by C. Jäger et al. (Proc. SPIE 4108, 104-110 (2001)) show that this compound is present in semicrystalline form, and there is thus the risk that it will (re)crystallize in the processed form, i.e. in the DSC.

In addition, the solubility in customary process solvents is relatively low, which leads to a correspondingly low degree of pore filling.

It was therefore an object of the present invention to provide further compounds which can be used advantageously as p-semiconductors in solar cells, especially in DSCs. With regard to their profile of properties, these compounds should have good hole-conducting properties, have only a very low tendency, if any, to crystallize, and have good solubility in the solvents used customarily, in order to bring about a maximum degree of filling of the oxide pores.

Accordingly, the use of the compounds cited at the outset in organic solar cells, and organic solar cells comprising these compounds, have been found.

Alkyl is understood to mean substituted or unsubstituted C₁-C₂₀-alkyl radicals. Preference is given to C₁- to C₁₀-alkyl radicals, particular preference to C₁- to C₈-alkyl radicals. The alkyl radicals may be either straight-chain or branched. In addition, the alkyl radicals may be substituted by one or more substituents selected from the group consisting of C₁-C₂₀-alkoxy, halogen, preferably F, and C₆-C₃₀-aryl which may in turn be substituted or unsubstituted. Examples of suitable alkyl groups are methyl, ethyl, propyl, butyl, pentyl, hexyl, heptyl and octyl, and also derivatives of the alkyl groups mentioned substituted by C₆-C₃₀-aryl, C₁-C₂₀-alkoxy and/or halogen, especially F, for example CF₃. Examples of linear and branched alkyl radicals are methyl, ethyl, propyl, butyl, pentyl, hexyl, heptyl and octyl, and also isopropyl, isobutyl, isopentyl, sec-butyl, tert-butyl, neopentyl, 3,3-dimethylbutyl, 2-ethylhexyl.

Divalent alkyl radicals in the A¹, A², A³, R, R′, R⁴, R⁵, R⁶, R⁷, R⁸ and R⁹ units derive from the aforementioned alkyl by formal removal of a further hydrogen atom.

Suitable aryls are C₆-C₃₀-aryl radicals which are derived from monocyclic, bicyclic or tricyclic aromatics and do not comprise any ring heteroatoms. When the aryls are not monocyclic systems, in the case of the term “aryl” for the second ring, the saturated form (perhydro form) or the partly unsaturated form (for example the dihydro form or tetrahydro form), provided the particular forms are known and stable, is also possible. The term “aryl” in the context of the present invention thus comprises, for example, also bicyclic or tricyclic radicals in which either both or all three radicals are aromatic, and also bicyclic or tricyclic radicals in which only one ring is aromatic, and also tricyclic radicals in which two rings are aromatic. Examples of aryl are: phenyl, naphthyl, indanyl, 1,2-dihydronaphthenyl, 1,4-dihydronaphthenyl, fluorenyl, indenyl, anthracenyl, phenanthrenyl or 1,2,3,4-tetrahydronaphthyl. Particular preference is given to C₆-C₁₀-aryl radicals, for example phenyl or naphthyl, very particular preference to C₆-aryl radicals, for example phenyl.

Aromatic groups in the A¹, A², A³, R, R′, R⁴, R⁵, R⁶, R⁷, R⁸ and R⁹ units derive from the aforementioned aryl by formal removal of one or more further hydrogen atoms.

Heteroaromatic groups in the A¹, A², A³, R, R′, R⁴, R⁵, R⁶, R⁷, R⁸ and R⁹ units derive from hetaryl radicals by formal removal of one or more further hydrogen atoms.

The parent hetaryl radicals here are unsubstituted or substituted and comprise 5 to 30 ring atoms. They may be monocyclic, bicyclic or tricyclic, and some can be derived from the aforementioned aryl by replacing at least one carbon atom in the aryl base skeleton with a heteroatom. Preferred heteroatoms are N, O and S. The hetaryl radicals more preferably have 5 to 13 ring atoms. The base skeleton of the heteroaryl radicals is especially preferably selected from systems such as pyridine and five-membered heteroaromatics such as thiophene, pyrrole, imidazole or furan. These base skeletons may optionally be fused to one or two six-membered aromatic radicals. Suitable fused heteroaromatics are carbazolyl, benzimidazolyl, benzofuryl, dibenzofuryl or dibenzothiophenyl. The base skeleton may be substituted at one, more than one or all substitutable positions, suitable substituents being the same as have already been specified under the definition of C₆-C₃₀-aryl. However, the hetaryl radicals are preferably unsubstituted. Suitable hetaryl radicals are, for example, pyridin-2-yl, pyridin-3-yl, pyridin-4-yl, thiophen-2-yl, thiophen-3-yl, pyrrol-2-yl, pyrrol-3-yl, furan-2-yl, furan-3-yl and imidazol-2-yl and the corresponding benzofused radicals, especially carbazolyl, benzimidazolyl, benzofuryl, dibenzofuryl or dibenzothiophenyl.

Possible further substituents of the one, two or three optionally substituted aromatic or heteroaromatic groups include alkyl radicals, for example methyl, ethyl, propyl, butyl, pentyl, hexyl, heptyl and octyl, and also isopropyl, isobutyl, isopentyl, sec-butyl, tert-butyl, neopentyl, 3,3-dimethylbutyl and 2-ethylhexyl, aryl radicals, for example C₆-C₁₀-aryl radicals, especially phenyl or naphthyl, most preferably C₆-aryl radicals, for example phenyl, and hetaryl radicals, for example pyridin-2-yl, pyridin-3-yl, pyridin-4-yl, thiophen-2-yl, thiophen-3-yl, pyrrol-2-yl, pyrrol-3-yl, furan-2-yl, furan-3-yl and imidazol-2-yl, and also the corresponding benzofused radicals, especially carbazolyl, benzimidazolyl, benzofuryl, dibenzofuryl or dibenzothiophenyl. The degree of substitution here may vary from monosubstitution up to the maximum number of possible substituents.

Preferred compounds of the formula I for use in accordance with the invention are notable in that at least two of the R¹, R² and R³ radicals are para-OR and/or -NR₂ substituents. The at least two radicals here may be only OR radicals, only NR₂ radicals, or at least one OR and at least one NR₂ radical.

Particularly preferred compounds of the formula I for use in accordance with the invention are notable in that at least four of the R¹, R² and R³ radicals are para-OR and/or -NR₂ substituents. The at least four radicals here may be only OR radicals, only NR₂ radicals or a mixture of OR and NR₂ radicals.

Very particularly preferred compounds of the formula I for use in accordance with the invention are notable in that all of the R¹, R² and R³ radicals are para-OR and/or -NR₂ substituents. They may be only OR radicals, only NR₂ radicals or a mixture of OR and NR₂ radicals.

In all cases, the two R in the NR₂ radicals may be different from one another, but they are preferably the same.

Preferred divalent organic A¹, A² and A³ units are selected from the group consisting of (CH₂)ₘ, C(R⁷)(R⁸), N(R⁹),

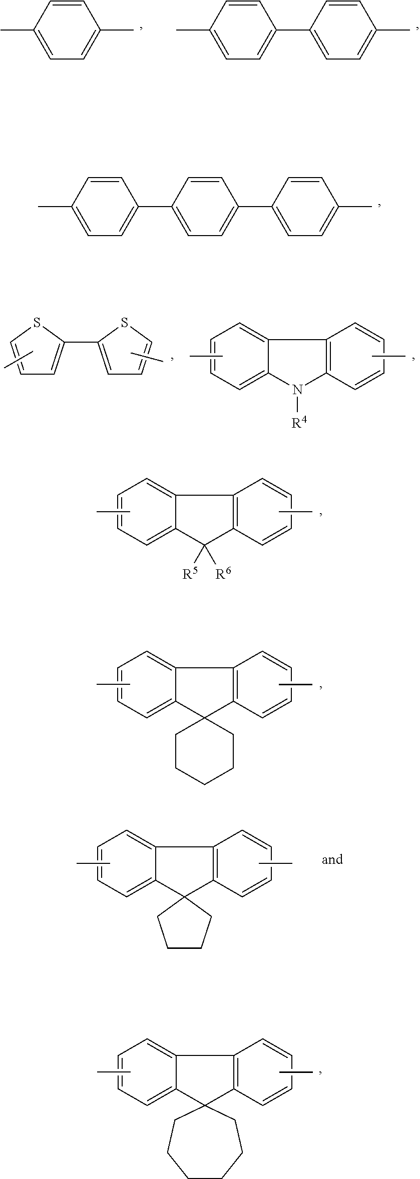

in which
m is an integer from 1 to 18,
R⁴, R⁹ are each alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R⁵, R⁶, R⁷, R⁸ are each independently hydrogen atoms or radicals as defined for R⁴ and R⁹,
   and the aromatic and heteroaromatic rings of the units shown may have further substitution.

The degree of substitution of the aromatic and heteroaromatic rings here may vary from monosubstitution up to the maximum number of possible substituents.

Preferred substituents in the case of further substitution of the aromatic and heteroaromatic rings include the substituents already mentioned above for the one, two or three optionally substituted aromatic or heteroaromatic groups.

The compounds for use in accordance with the invention can be prepared by customary methods of organic synthesis known to those skilled in the art. References to relevant (patent) literature references can additionally be found in the synthesis examples adduced below.

For the construction of a DSC, the n-semiconductive metal oxide used may be a single metal oxide or a mixture of different oxides. Use of mixed oxides is also possible. The n-semiconductive metal oxide can especially be used as a nanoparticulate oxide, nanoparticles in this connection being understood to mean particles which have an average particle size of less than 0.1 micrometer.

A nanoparticulate oxide is typically applied by a sintering process as a thin porous film with high surface area to a conductive substrate (i.e. a carrier with a conductive layer as the first electrode).

Suitable substrates (also referred to hereinafter as carriers) are, as well as metal foils, in particular polymer plates or films and especially glass plates. Suitable electrode materials, especially for the first electrode according to the above-described preferred structure, are especially conductive materials, for example transparent conducting oxides (TCOs), for example fluorine- and/or indium-doped tin oxide (FTO and ITO) and/or aluminum-doped zinc oxide (AZO), carbon nanotubes or metal films. Alternatively or additionally, however, it would also be possible to use thin metal films which still have sufficient transparency. The substrate can be covered or coated with these conductive materials.

Since only a single substrate is generally required in this construction, the construction of flexible cells is also possible. This enables a multitude of end uses which would be realizable with rigid substrates only with difficulty, if at all, for example use in bank cards, items of clothing, etc.

The first electrode, especially the TCO layer, may additionally be covered or coated with a solid buffer layer (for example of thickness 10 to 200 nm), especially a metal oxide buffer layer, in order to prevent direct contact of the p-semiconductor with the TCO layer (see Peng et al., Coord. Chem. Rev. 248, 1479 (2004)). The buffer metal oxide which can be used in the buffer layer may, for example, comprise one or more of the following materials: vanadium oxide; a zinc oxide; a tin oxide; a titanium oxide.

Thin layers or films of metal oxides are typically inexpensive solid semiconductor materials (n-semiconductors), but their absorption, owing to large band gaps, is usually not in the visible region of the solar spectrum, but predominantly in the ultraviolet spectral range. For use in solar cells, the metal oxides therefore generally, as is the case for the DSCs, have to be combined with a dye as a photosensitizer, which absorbs in the wavelength range of sunlight, i.e. at 300 to 2000 nm, and injects electrons into the charge band of the semiconductor in the electronically excited state. With the aid of a solid p-semiconductor used additionally in the cell, which is in turn reduced at the counterelectrode, electrons can be recycled to the sensitizer, such that it is regenerated.

Of particular interest for use in solar cells are the semiconductors zinc oxide, tin dioxide, titanium dioxide or mixtures of these metal oxides. The metal oxides can be used in the form of nanocrystalline porous layers. These layers have a large surface area which is coated with the sensitizer, such that a high absorption of sunlight is achieved. Metal oxide layers which are structured, for example nanorods, offer advantages such as higher electron mobilities or improved pore filling by the dye and the p-semiconductor.

The metal oxide semiconductors can be used alone or in the form of mixtures. It is also possible to coat a metal oxide with one or more other metal oxides. In addition, the metal oxides may also be applied as a coating on another semiconductor, for example GaP, ZnP or ZnS.

Particularly preferred semiconductors are zinc oxide and titanium dioxide in the anatase modification, which is preferably used in nanocrystalline form.

Moreover, the sensitizers can be combined advantageously with all n-semiconductors which typically find use in these solar cells. Preferred examples include metal oxides used in ceramics, such as titanium dioxide, zinc oxide, tin (IV) oxide, tungsten(VI) oxide, tantalum(V) oxide, niobium(V) oxide, cesium oxide, strontium titanate, zinc stannate, complex oxides of the perovskite type, for example barium titanate, and binary and ternary iron oxides, which may also be present in nanocrystalline or amorphous form.

Owing to the strong absorption that customary organic dyes and phthalocyanines and porphyrins have, even thin layers or films of the dye-sensitized n-semiconductive metal oxide are sufficient to achieve sufficient light absorption. Thin metal oxide films in turn have the advantage that the probability of undesired recombination processes falls and that the internal resistance of the dye subcell is reduced. For the n-semiconductive metal oxide, it is possible with preference to use layer thicknesses of 100 nm up to 20 micrometers, more preferably in the range between 500 nm and approx. 5 micrometers.

Numerous dyes which are usable in the context of the present invention are known from the prior art, such that reference may also be made to the above description of the prior art regarding dye solar cells for possible material examples. All dyes listed and claimed may in principle also be present as pigments. Dye-sensitized solar cells based on titanium dioxide as a semiconductor material are described, for example, in U.S. Pat. No. 4,927,721, Nature 353, p. 737-740 (1991) and U.S. Pat. No. 5,350,644, and also Nature 395, p. 583-585 (1998) and EP-A-1 176 646. The dyes described in these documents can in principle also be used advantageously in the context of the present invention. These dye solar cells comprise monomolecular films of transition metal complexes, especially ruthenium complexes, which are bound to the titanium dioxide layer via acid groups, as sensitizers.

Not least for reasons of cost, metal-free organic dyes have also been proposed repeatedly as sensitizers, and are also usable in the context of the present invention. High efficiencies of more than 4%, especially in solid-state dye solar cells, can be achieved, for example, with indoline dyes (see, for example, Schmidt-Mende et al., Adv. Mater. 2005, 17, 813). U.S. Pat. No. 6,359,211 describes the use, which is also usable in the context of the present invention, of cyanine, oxazine, thiazine and acridine dyes which have carboxyl groups bonded via an alkylene radical for fixing to the titanium dioxide semiconductor.

JP-A-10-189065, 2000-243463, 2001-093589, 2000-100484 and 10-334954 describe various perylene-3,4:9,10-tetracarboxylic acid derivatives unsubstituted in the perylene skeleton for use in semiconductor solar cells. These are specifically: perylenecarboximides which bear carboxyalkyl, carboxyaryl, carboxyarylalkyl or carboxyalkylaryl radicals on the imide nitrogen atoms, and/or are imidated with p-diaminobenzene derivatives, in which the nitrogen atom of the amino group is p-substituted by two further phenyl radicals or is part of a heteroaromatic tricyclic system; perylene-3,4:9,10-tetracarboxylic monoanhydride monoimides which bear the aforementioned radicals or alkyl or aryl radicals without further functionalization on the imide nitrogen atom, or semicondensates of perylene-3,4:9,10-tetracarboxylic dianhydride with 1,2-diaminobenzenes or 1,8-diaminonaphthalenes, which are converted by further reaction with primary amine to the corresponding diimides or double condensates; condensates of perylene-3,4:9,10-tetracarboxylic dianhydride with 1,2-diaminobenzenes, which are functionalized by carboxyl or amino radicals; and perylene-3,4:9,10-tetracarboximides which are imidated with aliphatic or aromatic diamines.

In New J. Chem. 26, p. 1155-1160 (2002), the sensitization of titanium dioxide with perylene derivatives unsubstituted in the perylene skeleton (bay positions) is studied. Specific examples mentioned are 9-dialkylaminoperylene-3,4-dicarboxylic anhydrides, perylene-3,4-dicarboximides which are 9-dialkylamino- or -carboxymethylamino-substituted and bear, on the imide nitrogen atom, a carboxymethyl or a 2,5-di(tert-butyl)phenyl radical, and N-dodecylaminoperylene-3,4:9,10-tetracarboxylic monoanhydride monoimide. However, the liquid electrolyte solar cells based on these perylene derivatives exhibited significantly lower efficiencies than a solar cell sensitized with a ruthenium complex for comparison.

Particularly preferred sensitizer dyes in the dye solar cell proposed are the perylene derivatives, terrylene derivatives and quaterrylene derivatives described in DE 10 2005 053 995 A1 or WO 2007/054470 A1. The use of these dyes leads to photovoltaic elements with high efficiencies and simultaneously high stabilities.

The rylenes exhibit strong absorption in the wavelength range of sunlight and may, depending on the length of the conjugated system, cover a range from about 400 nm (perylene derivatives I from DE 10 2005 053 995 A1) up to about 900 nm (quaterrylene derivatives I from DE 10 2005 053 995 A1). Rylene derivatives I based on terrylene absorb, according to their composition, in the solid state adsorbed on titanium dioxide, within a range from about 400 to 800 nm. In order to achieve maximum utilization of the incident sunlight from the visible up to the near infrared range, it is advantageous to use mixtures of different rylene derivatives I. Occasionally, it may also be advisable to use different rylene homologs.

The rylene derivatives I can be fixed easily and in a durable manner on the metal oxide film. The binding is effected via the anhydride function (×1) or the carboxyl groups —COOH or —COO— formed in situ, or via the acid groups A present in the imide or condensate radicals ((×2) or (×3)). The rylene derivatives I described in DE 10 2005 053 995 A1 are very suitable for use in dye-sensitized solar cells in the context of the present invention.

The dyes more preferably have an anchor group at one end of the molecule, which ensures their fixing on the n-semiconductor film. At the other end of the molecule, the dyes preferably comprise electron donors which facilitate the regeneration of the dye after the electron release to the n-semiconductor, and also prevent recombination with electrons already released to the semiconductor.

For further details regarding the possible selection of a suitable dye, reference may be made, for example, again to DE 10 2005 053 995 A1. For the solid state dye solar cells described in the present document, especially ruthenium complexes, porphyrins, other organic sensitizers and preferably rylenes can be used.

The dyes can be fixed on the metal oxide films in a simple manner. For example, the n-semiconductive metal oxide films in the freshly sintered (still warm) state can be contacted with a solution or suspension of the dye in a suitable organic solvent over a sufficient period (e.g. about 0.5 to 24 h). This can be done, for example, by immersing the substrate coated with the metal oxide into the solution of the dye.

If combinations of different dyes are to be used, they can be applied successively, for example, from one or more solutions or suspensions which comprise one or more of the dyes. It is also possible to use two dyes which are separated by a layer of, for example, CuSCN (on this subject, see, for example, Tennakone, K. J., Phys. Chem. B. 2003, 107, 13758). The most appropriate method can be determined comparatively easily in the individual case.

In principle, the dye may be present as a separate element or may be applied in a separate step and applied separately to the remaining layers. Alternatively or additionally, the dye may, however, also be combined or applied together with one or more of the other elements, for example with the solid p-semiconductor. For example, a dye-p-semiconductor combination which comprises an absorbent dye with p-semiconductive properties or, for example, a pigment with absorbent and p-semiconductive properties can be used.

In order to prevent recombination of the electrons in the n-semiconductive metal oxide with the solid p-conductor, a kind of passivating layer which comprises a passivation material can be used. This layer should be as thin as possible and should as far as possible cover only the sites on the n-semiconductive metal oxide which are as yet uncovered. Under some circumstances, the passivation material may also be applied to the metal oxide before the dye. Preferred passivation materials are especially the following substances: Al₂O₃; an aluminum salt; silanes, for example CH₃SiCl₃; an organometallic complex, especially an Al³⁺ complex; Al³⁺, especially an Al³⁺ complex; 4-tert-butylpyridine (TBP); MgO; 4-guanidinobutanoic acid (GBA); an alkanoic acid; hexadecylmalonic acid (HDMA).

As already mentioned above, solid p-semiconductors are used in the solid state dye solar cell. Solid p-semiconductors can also be used in the inventive dye-sensitized solar cells without any great increase in the cell resistance, especially when the dyes have strong absorption and therefore require only thin n-semiconductor layers. More particularly, the p-semiconductor should essentially have a continuous, impervious layer in order that undesired recombination reactions which could arise from contact between the n-semiconductive metal oxide (especially in nanoporous form) with the second electrode or the second half-cell are reduced.

A significant parameter influencing the selection of the p-semiconductor is the hole mobility, since this partly determines the hole diffusion length (cf. Kumara, G., Langmuir, 2002, 18, 10493-10495). A comparison of charge carrier mobilities in various Spiro compounds can be found, for example, in Saragi, T., Adv. Funct. Mater. 2006, 16, 966-974.

In addition, reference is made to the remarks regarding the p-semiconductive materials in the description of the prior art.

With regard to the remaining possible construction elements and the possible construction of the dye solar cell, reference is likewise made to the description above.

### EXAMPLES

### Synthesis of the Compounds of the Formula I for Use in Accordance with the Invention

### A) Syntheses:

Synthesis route I:

### Synthesis Step I-R1:

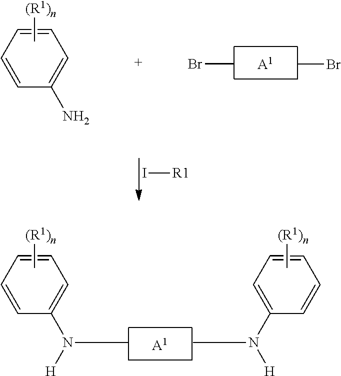

The synthesis in synthesis step I-R1 was effected based on the literature references cited below:
a) Liu, Yunqi; Ma, Hong; Jen, Alex K-Y.; CHCOFS; Chem. Commun.; 24; 1998; 2747-2748,
b) Goodson, Felix E.; Hauck, Sheila; Hartwig, John F.; J. Am. Chem. Soc.; 121; 33; 1999; 7527-7539,
c) Shen, Jiun Yi; Lee, Chung Ying; Huang, Tai-Hsiang; Lin, Jiann T.; Tao, Yu-Tai; Chien, Chin-Hsiung; Tsai, Chiitang; J. Mater. Chem.; 15; 25; 2005; 2455-2463,
d) Huang, Ping-Hsin; Shen, Jiun-Yi; Pu, Shin-Chien; Wen, Yuh-Sheng; Lin, Jiann T.; Chou, Pi-Tai; Yeh, Ming-Chang P.; J. Mater. Chem.; 16; 9; 2006; 850-857,
e) Hirata, Narukuni; Kroeze, Jessica E.; Park, Taiho; Jones, David; Haque, Saif A.; Holmes, Andrew B.; Durrant, James R.; Chem. Commun.; 5; 2006; 535-537.

### Synthesis Step I-R2:

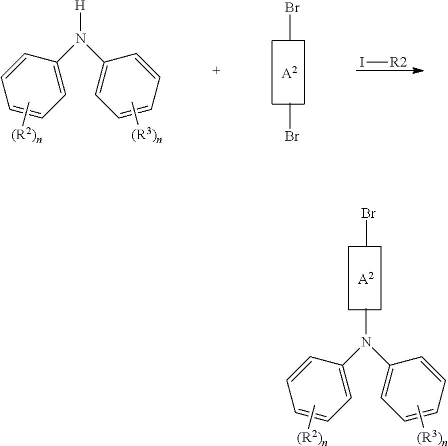

The synthesis in synthesis step I-R2 was effected based on the literature references cited below:
a) Huang, Qinglan; Evmenenko, Guennadi; Dutta, Pulak; Marks, Tobin J.; J. Am. Chem. Soc.; 125; 48; 2003; 14704-14705,
b) Bacher, Erwin; Bayerl, Michael; Rudati, Paula; Reckefuss, Nina; Mueller, C. David; Meerholz, Klaus; Nuyken, Oskar; Macromolecules; EN; 38; 5; 2005; 1640-1647,
c) Li, Zhong Hui; Wong, Man Shing; Tao, Ye; D'Iorio, Marie; J. Org. Chem.; EN; 69; 3; 2004; 921-927.

### Synthesis Step I-R3:

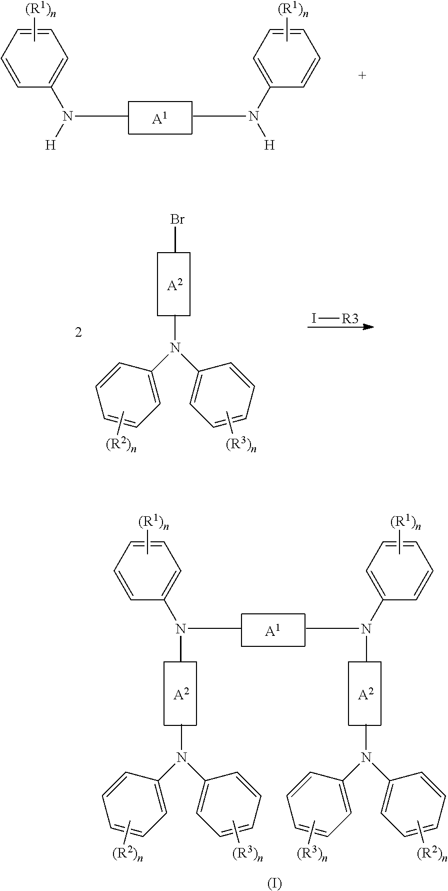

The synthesis in synthesis step I-R3 was effected based on the literature reference cited below:
J. Grazulevicius; J. of Photochem. and Photobio., A: Chemistry 2004 162(2-3), 249-252.

The compounds of the formula I can be prepared via the sequence of the synthesis steps of synthesis route I shown above. The reactants can be coupled, for example, by Ullmann reaction with copper as a catalyst or with palladium catalysis.

Synthesis route II:

### Synthesis Step II-R1:

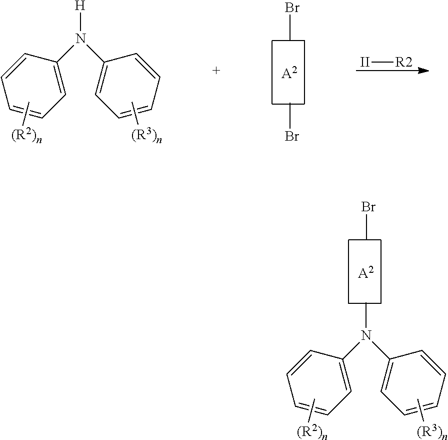

The synthesis in synthesis step II-R1 was effected based on the literature references cited under II-R2.

### Synthesis Step II-R2:

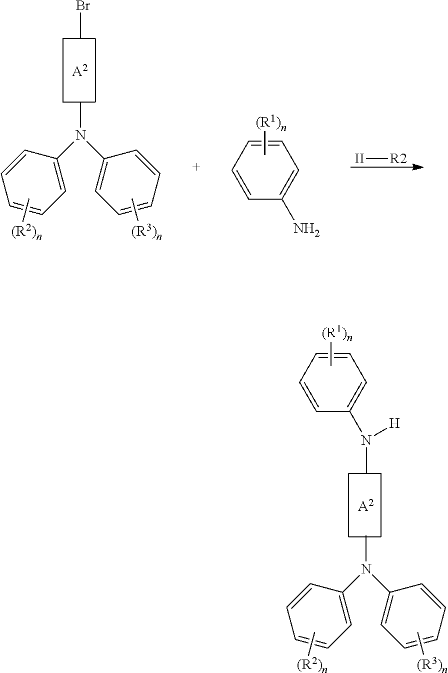

The synthesis in synthesis step II-R2 was effected based on the literature references cited below:
a) Bacher, Erwin; Bayerl, Michael; Rudati, Paula; Reckefuss, Nina; Müller, C. David; Meerholz, Klaus; Nuyken, Oskar; Macromolecules; 38; 5; 2005; 1640-1647,
b) Goodson, Felix E.; Hauck, Sheila; Hartwig, John F.; J. Am. Chem. Soc.; 121; 33; 1999; 7527-7539; Hauck, Sheila I.; Lakshmi, K. V.; Hartwig, John F.; Org. Lett.; 1; 13; 1999; 2057-2060.

### Synthesis Step II-R3:

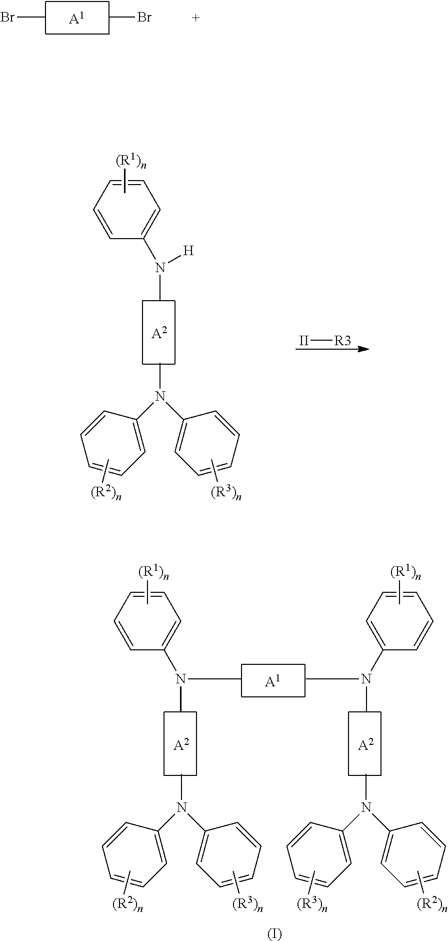

The compounds of the formula I can be prepared via the sequence of the synthesis steps of synthesis route II shown above. The reactants can be coupled, as also in synthesis route I, for example, by Ullmann reaction with copper as a catalyst or with palladium catalysis.

### Preparation of the Starting Amines:

When the diarylamines in synthesis steps I-R2 and II-R1 of synthesis routes I and II are not commercially available, they can be prepared, for example, by Ullmann reaction with copper as a catalyst or under palladium catalysis, according to the following reaction:

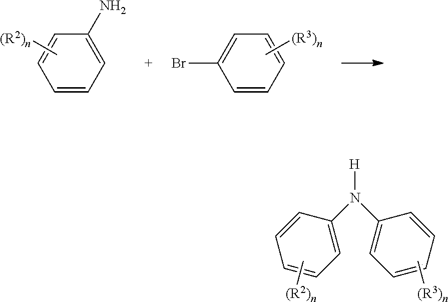

The synthesis was effected based on the review articles listed below:

Palladium-catalyzed C—N coupling reactions:
a) Yang, Buchwald; J. Organomet. Chem. 1999, 576 (1-2), 125-146,
b) Wolfe, Marcoux, Buchwald; Acc. Chem. Res. 1998, 31, 805-818,
c) Hartwig; Angew. Chem. Int. Ed. Engl. 1998, 37, 2046-2067.

Copper-catalyzed C—N coupling reactions:
a) Goodbrand, Hu; Org. Chem. 1999, 64, 670-674,
b) Lindley; Tetrahedron 1984, 40, 1433-1456.

### Example 1

### Synthesis of the Compound ID367

### Synthesis Route I

### Synthesis Step I-R1:

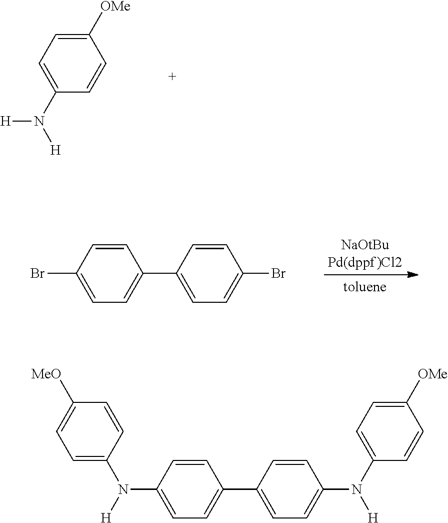

A mixture of 4,4′-dibromobiphenyl (93.6 g; 300 mmol), 4-methoxyaniline (133 g; 1.08 mol), Pd(dppf)Cl₂ (Pd(1,1′-bis(diphenylphosphino)ferrocene)Cl₂; 21.93 g; 30 mmol) and t-BuONa (sodium tert-butoxide; 109.06 g; 1.136 mol) in toluene (1500 ml) was stirred under a nitrogen atmosphere at 110° C. for 24 hours. After cooling, the mixture was diluted with diethyl ether and filtered through a Celite® pad (from Carl Roth). The filter bed was washed with 1500 ml each of ethyl acetate, methanol and methylene chloride. The product was obtained as a light brown solid (36 g; yield: 30%).

¹HNMR (400 MHz, DMSO): δ 7.81 (s, 2H), 7.34-7.32 (m, 4H), 6.99-6.97 (m, 4H), 6.90-6.88 (m, 4H), 6.81-6.79 (m, 4H), 3.64 (s, 6H).

### Synthesis Step I-R2:

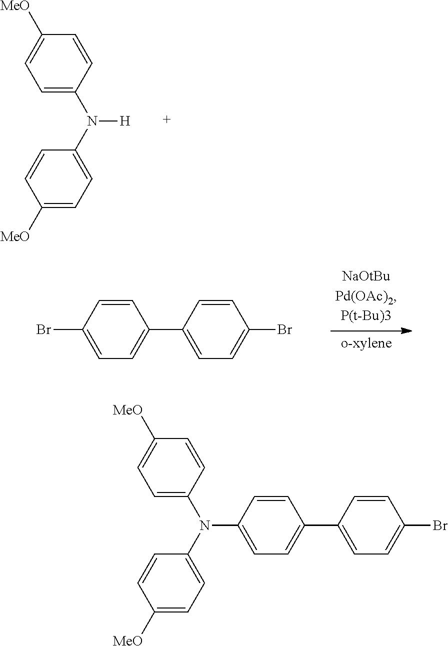

Nitrogen was passed for a period of 10 minutes through a solution of dppf (1,1′-bis(diphenylphosphino)ferrocene; 0.19 g; 0.34 mmol) and Pd₂(dba)₃(tris(dibenzylideneacetone)dipalladium(0); 0.15 g; 0.17 mmol) in toluene (220 ml). Subsequently, t-BuONa (2.8 g; 29 mmol) was added and the reaction mixture was stirred for a further 15 minutes. 4,4′-Dibromobiphenyl (25 g; 80 mmol) and 4,4′-dimethoxydiphenylamine (5.52 g; 20 mmol) were then added successively. The reaction mixture was heated at a temperature of 100° C. under a nitrogen atmosphere for 7 hours. After cooling to room temperature, the reaction mixture was quenched with ice-water, and the precipitated solid was filtered off and dissolved in ethyl acetate. The organic layer was washed with water, dried over sodium sulfate and purified by column chromatography (eluent: 5% ethyl acetate/hexane). A pale yellow-colored solid was obtained (7.58 g, yield: 82%).

¹HNMR (300 MHz, DMSO-d₆): 7.60-7.49 (m, 6H), 7.07-7.04 (m, 4H), 6.94-6.91 (m, 4H), 6.83-6.80 (d, 2H), 3.75 (s, 6H).

### Synthesis Step I-R3:

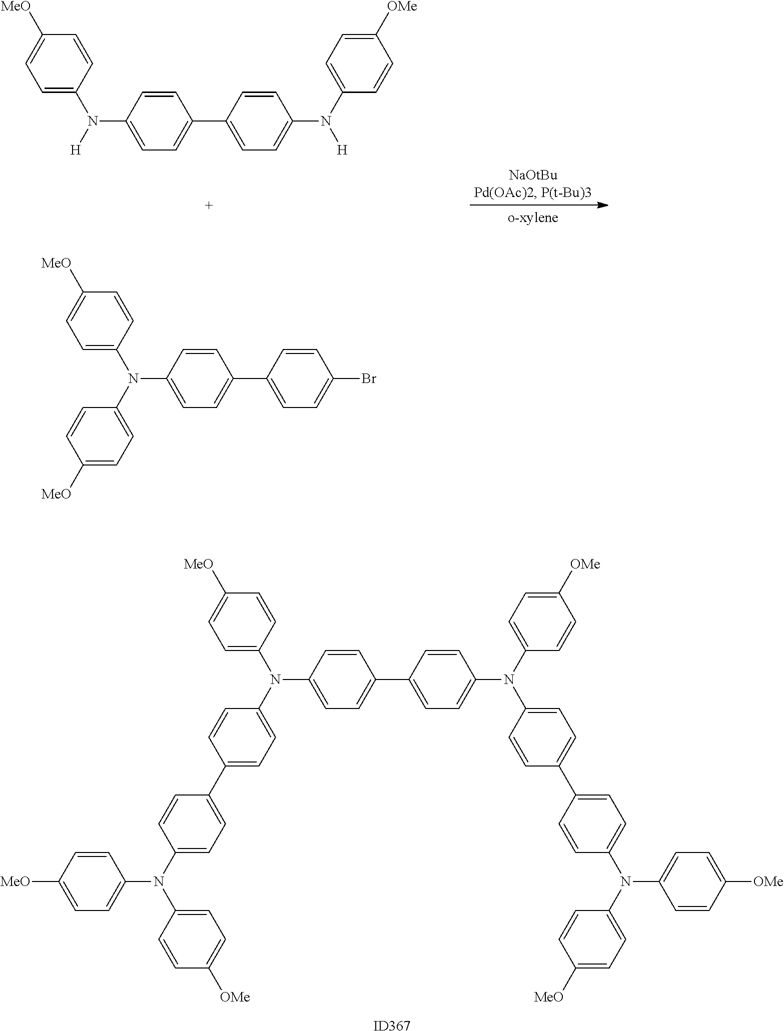

N⁴,N^{4′}-Bis(4-methoxyphenyl)biphenyl-4,4′-diamine (product from synthesis step I-R1; 0.4 g; 1.0 mmol) and product from synthesis step I-R2 (1.0 g; 2.2 mmol) were added under a nitrogen atmosphere to a solution of t-BuONa (0.32 g; 3.3 mmol) in o-xylene (25 ml). Subsequently, palladium acetate (0.03 g; 0.14 mmol) and a solution of 10% by weight of P(t-Bu)₃ (tris-t-butylphosphine) in hexane (0.3 ml; 0.1 mmol) were added to the reaction mixture which was stirred at 125° C. for 7 hours. Thereafter, the reaction mixture was diluted with 150 ml of toluene and filtered through Celite®, and the organic layer was dried over Na₂SO₄. The solvent was removed and the crude product was reprecipitated three times from a mixture of tetrahydrofuran (THF)/methanol. The solid was purified by column chromatography (eluent: 20% ethyl acetate/hexane), followed by a precipitation with THF/methanol and an activated carbon purification. After removing the solvent, the product was obtained as a pale yellow solid (1.0 g, yield: 86%).

¹HNMR (400 MHz, DMSO-d₆): 7.52-7.40 (m, 8H), 6.88-7.10 (m, 32H), 6.79-6.81 (d, 4H), 3.75 (s, 6H), 3.73 (s, 12H).

### Example 2

### Synthesis of the Compound ID447

### Synthesis Route II

### Synthesis Step I-R1:

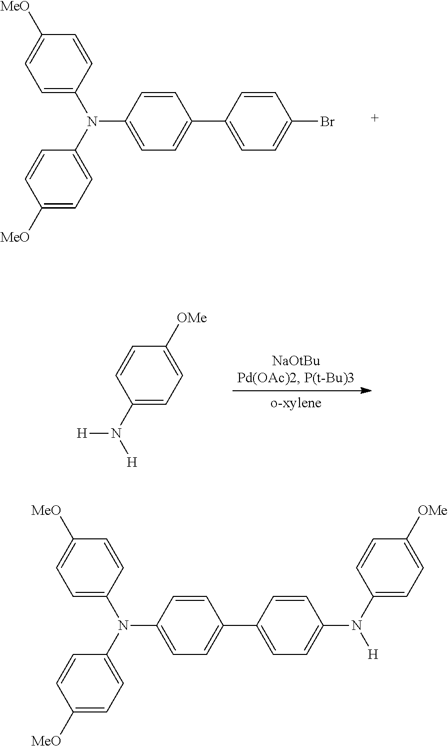

p-Anisidine (5.7 g, 46.1 mmol), t-BuONa (5.5 g, 57.7 mmol) and P(t-Bu)₃ (0.62 ml, 0.31 mmol) were added to a solution of the product from synthesis step I-R2 (17.7 g, 38.4 mmol) in toluene (150 ml). After nitrogen had been passed through the reaction mixture for 20 minutes, Pd₂(dba)₃ (0.35 g, 0.38 mmol) was added. The resulting reaction mixture was left to stir under a nitrogen atmosphere at room temperature for 16 hours. Subsequently, it was diluted with ethyl acetate and filtered through Celite®. The filtrate was washed twice with 150 ml each of water and saturated sodium chloride solution. After the organic phase had been dried over Na₂SO₄ and the solvent had been removed, a black solid was obtained. This solid was purified by column chromatography (eluent: 0-25% ethyl acetate/hexane). This afforded an orange solid (14 g, yield: 75%).

¹HNMR (300 MHz, DMSO): 7.91 (s, 1H), 7.43-7.40 (d, 4H), 7.08-6.81 (m, 16H), 3.74 (s, 6H), 3.72 (s, 3H).

### Synthesis Step I-R3:

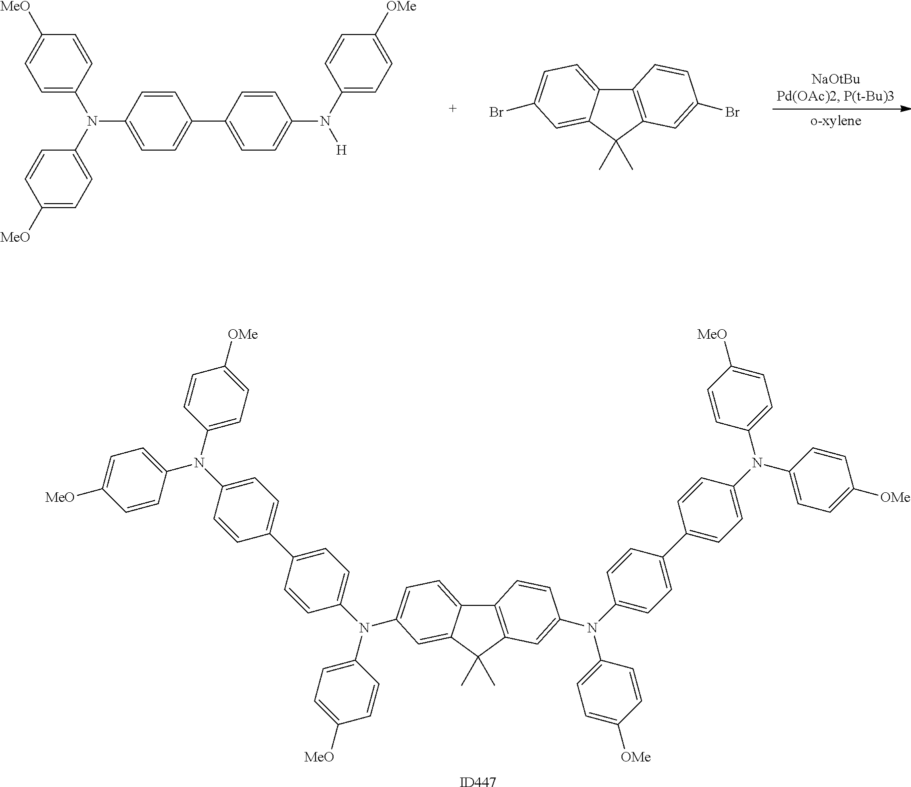

t-BuONa (686 mg; 7.14 mmol) was heated at 100° C. under reduced pressure, then the reaction flask was purged with nitrogen and allowed to cool to room temperature. 2,7-Dibromo-9,9-dimethylfluorene (420 mg; 1.19 mmol), toluene (40 ml) and Pd[P(^{t}Bu)₃]₂ (20 mg; 0.0714 mmol) were then added, and the reaction mixture was stirred at room temperature for 15 minutes. Subsequently, N,N,N′-p-trimethoxytriphenylbenzidine (1.5 g; 1.27 mmol) was added to the reaction mixture which was stirred at 120° C. for 5 hours. The mixture was filtered through a Celite®/MgSO₄ mixture and washed with toluene. The crude product was purified twice by column chromatography (eluent: 30% ethyl acetate/hexane) and, after twice reprecipitating from THF/methanol, a pale yellow-colored solid was obtained (200 mg, yield: 13%).

¹H NMR: (400 MHz, DMSO-d₆): 7.60-7.37 (m, 8H), 7.02-6.99 (m, 16H), 6.92-6.87 (m, 20H), 6.80-6.77 (d, 2H), 3.73 (s, 6H), 3.71 (s, 12H), 1.25 (s, 6H)

### Example 3

### Synthesis of the Compound ID453

### Synthesis Route I

### a) Preparation of the Starting Amine:

### Step 1:

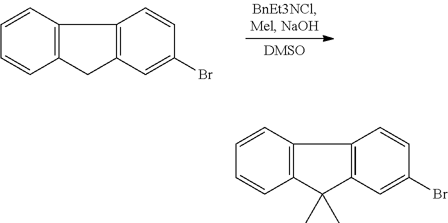

NaOH (78 g; 4 eq) was added to a mixture of 2-bromo-9H-fluorene (120 g; 1 eq) and BnEt₃NCl (benzyltriethylammonium chloride; 5.9 g; 0.06 eq) in 580 ml of DMSO (dimethyl sulfoxide). The mixture was cooled with ice-water, and methyl iodide (MeI) (160 g; 2.3 eq) was slowly added dropwise. The reaction mixture was left to stir overnight, then poured into water and subsequently extracted three times with ethyl acetate. The combined organic phases were washed with a saturated sodium chloride solution and dried over Na2SO4, and the solvent was removed. The crude product was purified by column chromatography using silica gel (eluent: petroleum ether). After washing with methanol, the product (2-bromo-9,9′-dimethyl-9H-fluorene) was obtained as a white solid (102 g).

¹HNMR (400 MHz, CDCl3): δ 1.46 (s, 6H), 7.32 (m, 2H), 7.43 (m, 2H), 7.55 (m, 2H), 7.68 (m, 1H)

### Step 2:

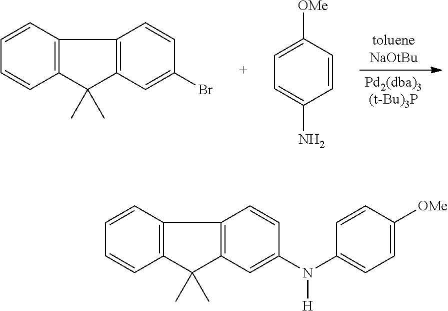

p-Anisidine (1.23 g; 10.0 mmol) and 2-bromo-9,9′-dimethyl-9H-fluorene (3.0 g; 11.0 mmol) were added under a nitrogen atmosphere to a solution of t-BuONa (1.44 g; 15.0 mmol) in 15 ml of toluene. Pd₂(dba)₃ (92 mg; 0.1 mmol) and a 10% by weight solution of P(t-Bu)₃ in hexane (0.24 ml; 0.08 mmol) were added, and the reaction mixture was stirred at room temperature for 5 hours. Subsequently, the mixture was quenched with ice-water, and the precipitated solid was filtered off and dissolved in ethyl acetate. The organic phase was washed with water and dried over Na₂SO₄. After purifying the crude product by column chromatography (eluent: 10% ethyl acetate/hexane), a pale yellow-colored solid was obtained (1.5 g, yield: 48%).

¹HNMR (300 MHz, C₆D₆): 7.59-7.55 (d, 1H), 7.53-7.50 (d, 1H), 7.27-7.22 (t, 2H), 7.19 (s, 1H), 6.99-6.95 (d, 2H), 6.84-6.77 (m, 4H), 4.99 (s, 1H), 3.35 (s, 3H), 1.37 (s, 6H).

b) Preparation of the Compound for Use in Accordance with the Invention

### Synthesis Step I-R2:

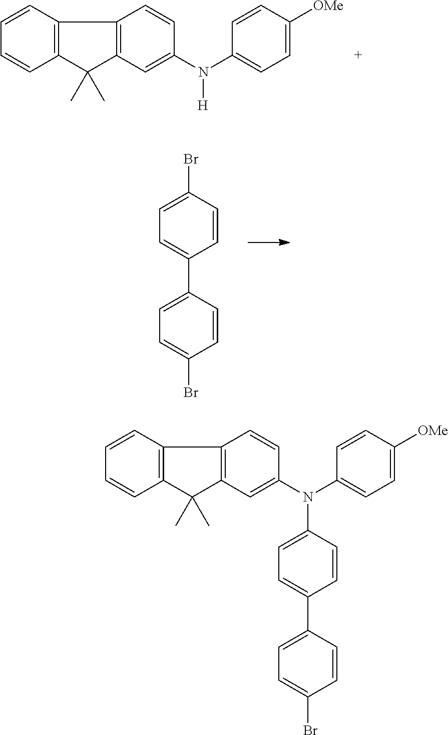

Product from a) (4.70 g; 10.0 mmol) and 4,4′-dibromobiphenyl (7.8 g; 25 mmol) were added to a solution of t-BuONa (1.15 g; 12 mmol) in 50 ml of toluene under nitrogen. Pd₂(dba)₃ (0.64 g; 0.7 mmol) and DPPF (0.78 g; 1.4 mmol) were added, and the reaction mixture was left to stir at 100° C. for 7 hours. After the reaction mixture had been quenched with ice-water, the precipitated solid was filtered off and it was dissolved in ethyl acetate. The organic phase was washed with water and dried over Na₂SO₄. After purifying the crude product by column chromatography (eluent: 1% ethyl acetate/hexane), a pale yellow-colored solid was obtained (4.5 g, yield: 82%).

¹1HNMR (400 MHz, DMSO-d6): 7.70-7.72 (d, 2H), 7.54-7.58 (m, 6H), 7.47-7.48 (d, 1H), 7.21-7.32 (m, 3H), 7.09-7.12 (m, 2H), 6.94-6.99 (m, 4H), 3.76 (s, 3H), 1.36 (s, 6H).

### Synthesis Step I-R3:

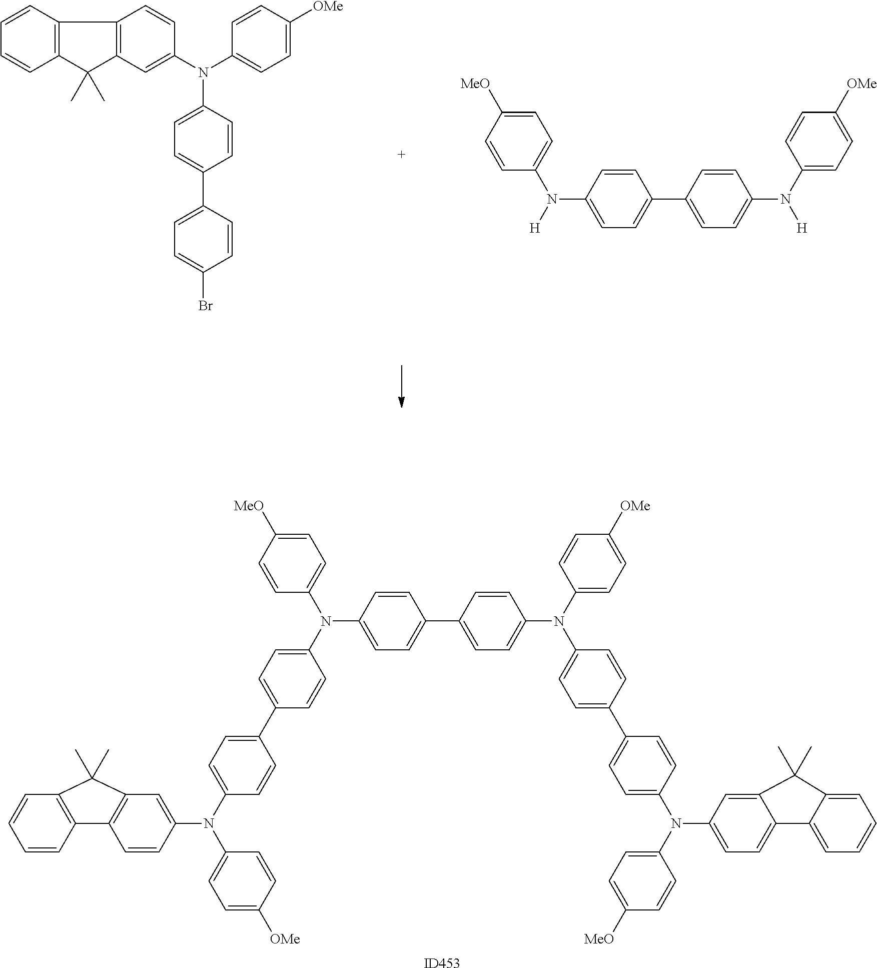

N⁴,N^{4′}-Bis(4-methoxyphenyl)biphenyl-4,4′-diamine (0.60 g; 1.5 mmol) and product from the preceding synthesis step I-R2 (1.89 g; 3.5 mmol) were added under nitrogen to a solution of t-BuONa (0.48 g; 5.0 mmol) in 30 ml of o-xylene. Palladium acetate (0.04 g; 0.18 mmol) and P(t-Bu)₃ in a 10% by weight solution in hexane (0.62 ml; 0.21 mmol) were added, and the reaction mixture was stirred at 125° C. for 6 hours. Subsequently, the mixture was diluted with 100 ml of toluene and filtered through Celite®. The organic phase was dried over Na₂SO₄ and the resulting solid was purified by column chromatography (eluent: 10% ethyl acetate/hexane). This was followed by reprecipitation from THF/methanol to obtain a pale yellow-colored solid (1.6 g, yield: 80%).

¹HNMR (400 MHz, DMSO-d₆): 7.67-7.70 (d, 4H), 7.46-7.53 (m, 14H), 7.21-7.31 (m, 4H), 7.17-7.18 (d, 2H), 7.06-7.11 (m, 8H), 6.91-7.01 (m, 22H), 3.75 (s, 12H), 1.35 (s, 12H).

Further compounds of the formula I for use in accordance with the invention:

The compounds listed below were obtained analogously to the syntheses described above:

### Example 4

### Compound ID320

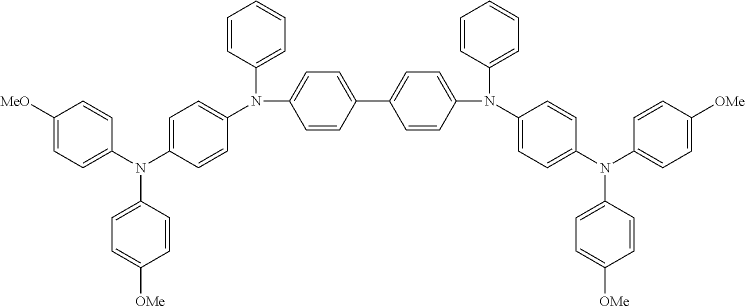

¹HNMR (300 MHz, THF-d₈): δ 7.43-7.46 (d, 4H), 7.18-7.23 (t, 4H), 7.00-7.08 (m, 16H), 6.81-6.96 (m, 18H), 3.74 (s, 12H)

### Example 5

### Compound ID321

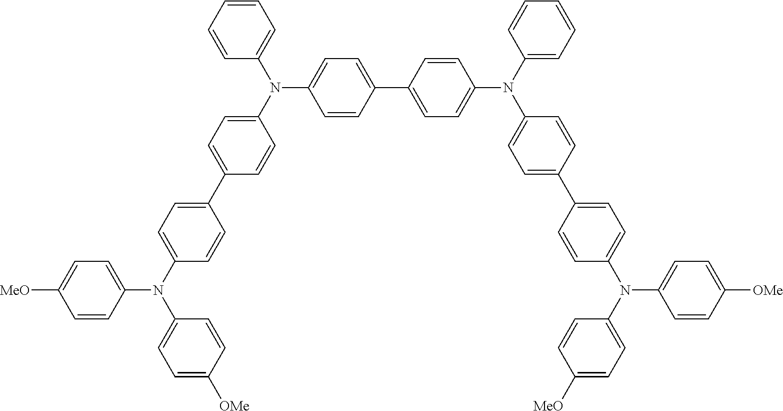

¹HNMR (300 MHz, THF-d₈): δ 7.37-7.50 (t, 8H), 7.37-7.40 (d, 4H), 7.21-7.26 (d, 4H), 6.96-7.12 (m, 22H), 6.90-6.93 (d, 4H), 6.81-6.84 (d, 8H), 3.74 (s, 12H)

### Example 6

### Compound ID366

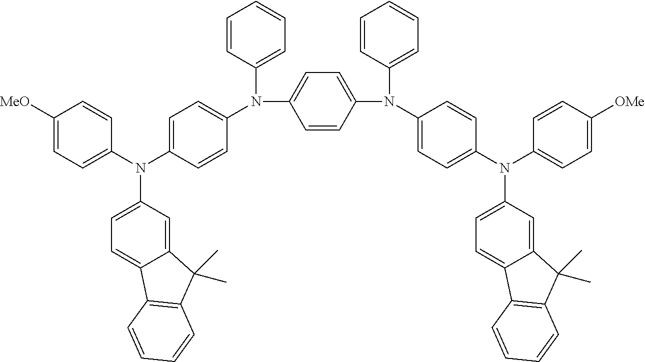

¹HNMR (400 MHz, DMSO-d6): δ 7.60-7.70 (t, 4H), 7.40-7.55 (d, 2H), 7.17-7.29 (m, 8H), 7.07-7.09 (t, 4H), 7.06 (s, 2H), 6.86-7.00 (m, 24H), 3.73 (s, 6H), 1.31 (s, 12H)

### Example 7

### Compound ID368

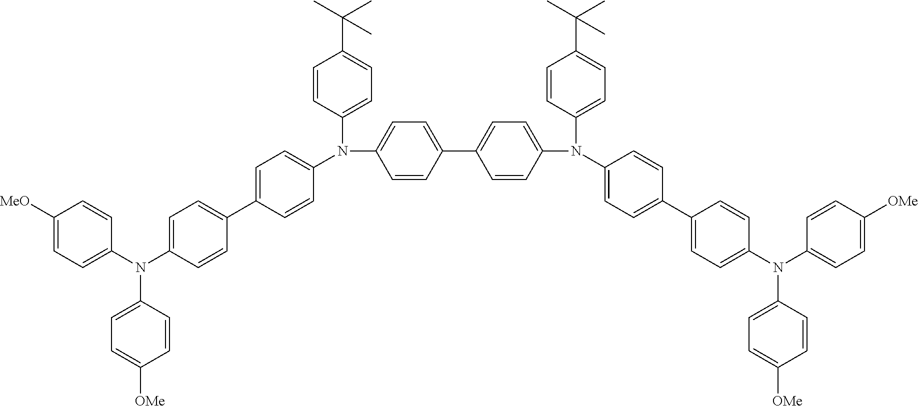

¹HNMR (400 MHz, DMSO-d6): δ 7.48-7.55 (m, 8H), 7.42-7.46 (d, 4H), 7.33-7.28 (d, 4H), 6.98-7.06 (m, 20H), 6.88-6.94 (m, 8H), 6.78-6.84 (d, 4H), 3.73 (s, 12H), 1.27 (s, 18H)

### Example 8

### Compound ID369

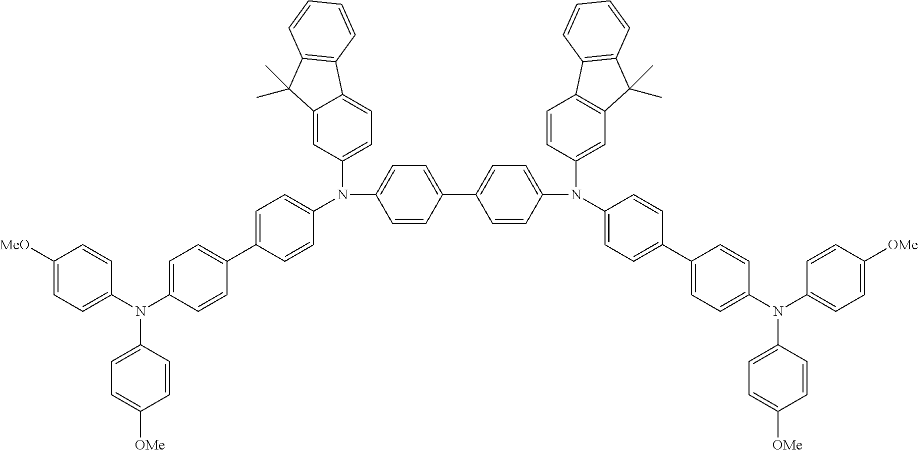

¹HNMR (400 MHz, THF-d8): δ 7.60-7.70 (t, 4H), 7.57-7.54 (d, 4H), 7.48-7.51 (d, 4H), 7.39-7.44 (t, 6H), 7.32-7.33 (d, 2H), 7.14-7.27 (m, 12H), 7.00-7.10 (m, 10H), 6.90-6.96 (m, 4H), 6.80-6.87 (m, 8H), 3.75 (s, 12H), 1.42 (s, 12H)

### Example 9

### Compound ID446

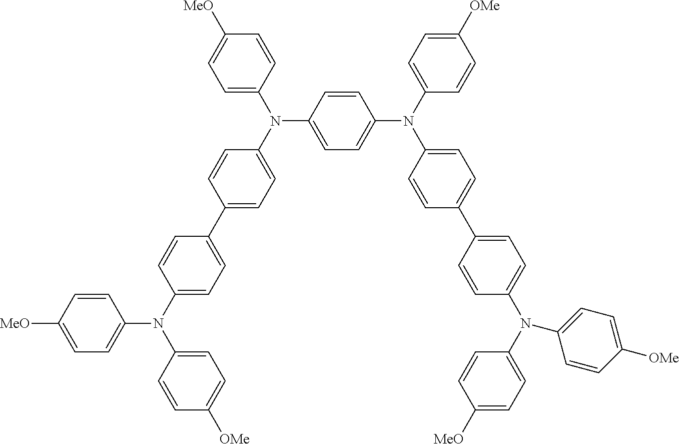

¹HNMR (400 MHz, dmso-d₆): δ 7.39-7.44 (m, 8H), 7.00-7.07 (m, 13H), 6.89-6.94 (m, 19H), 6.79-6.81 (d, 4H), 3.73 (s, 18H)

### Example 10

### Compound ID450

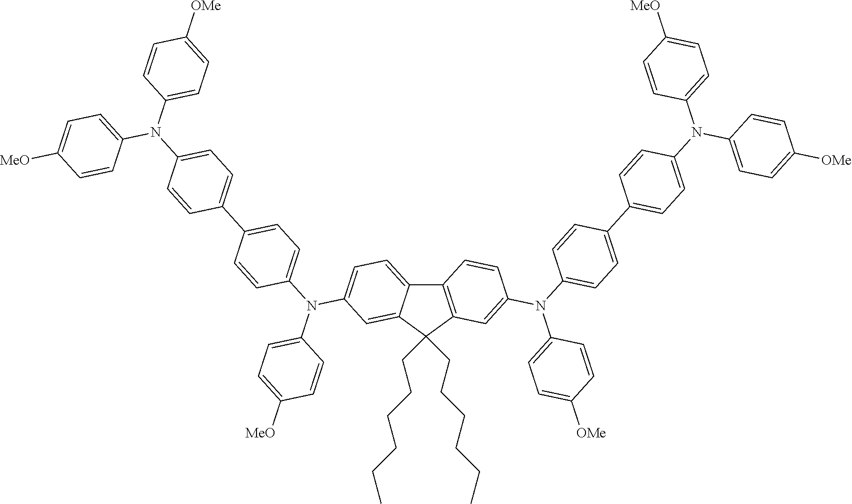

¹HNMR (400 MHz, dmso-d₆): δ 7.55-7.57 (d, 2H), 7.39-7.45 (m, 8H), 6.99-7.04 (m, 15H), 6.85-6.93 (m, 19H), 6.78-6.80 (d, 4H), 3.72 (s, 18H), 1.68-1.71 (m, 6H), 1.07 (m, 6H), 0.98-0.99 (m, 8H), 0.58 (m, 6H)

### Example 11

### Compound ID452

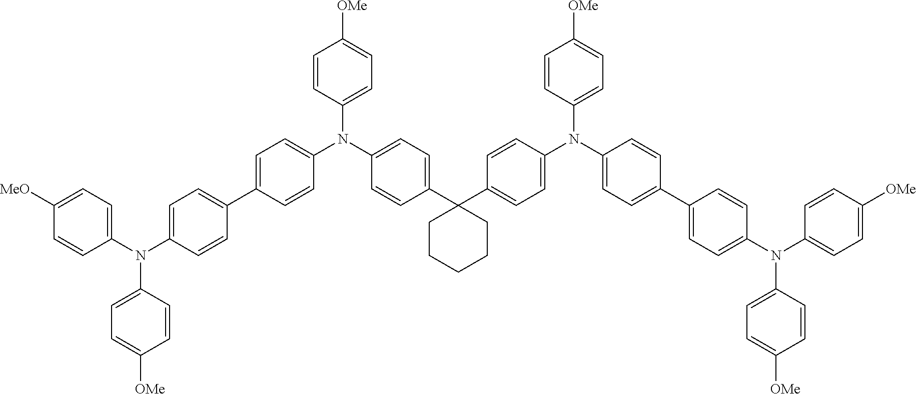

¹HNMR (400 MHz, DMSO-d6): δ 7.38-7.44 (m, 8H), 7.16-7.19 (d, 4H), 6.99-7.03 (m, 12H), 6.85-6.92 (m, 20H), 6.77-6.79 (d, 4H), 3.74 (s, 18H), 2.00-2.25 (m, 4H), 1.25-1.50 (m, 6H)

### Example 12

### Compound ID480

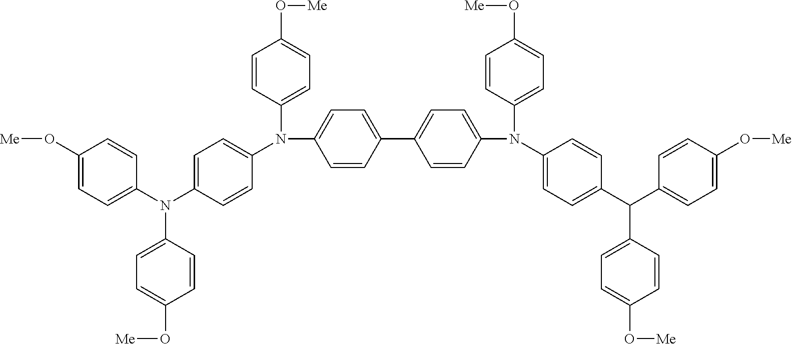

¹HNMR (400 MHz, DMSO-d6): δ 7.40-7.42 (d, 4H), 7.02-7.05 (d, 4H), 6.96-6.99 (m, 28H), 6.74-6.77 (d, 4H), 3.73 (s, 6H), 3.71 (s, 12H)

### Example 13

### Compound ID518

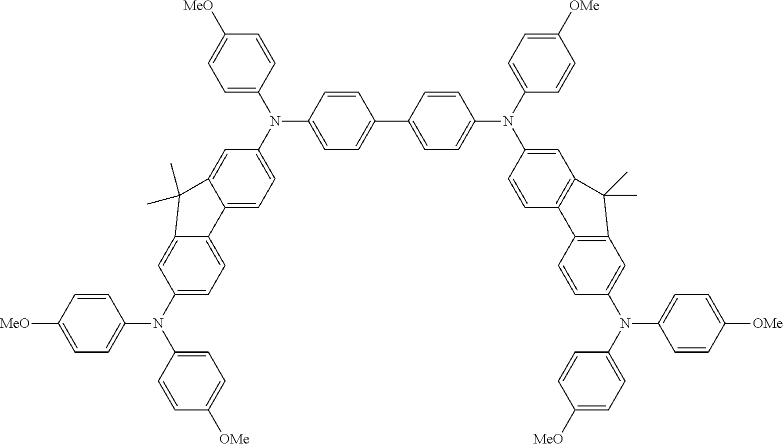

¹HNMR (400 MHz, DMSO-d6): 7.46-7.51 (m, 8H), 7.10-7.12 (d, 2H), 7.05-7.08 (d, 4H), 6.97-7.00 (d, 8H), 6.86-6.95 (m, 20H), 6.69-6.72 (m, 2H), 3.74 (s, 6H), 3.72 (s, 12H), 1.24 (t, 12H)

### Example 14

### Compound ID519

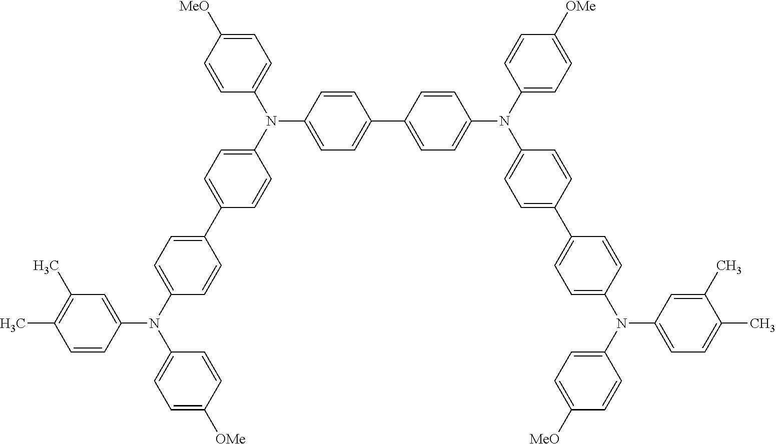

¹HNMR (400 MHz, DMSO-d6): 7.44-7.53 (m, 12H), 6.84-7.11 (m, 32H), 6.74-6.77 (d, 2H), 3.76 (s, 6H), 3.74 (s, 6H), 2.17 (s, 6H), 2.13 (s, 6H)

### Example 15

### Compound ID521

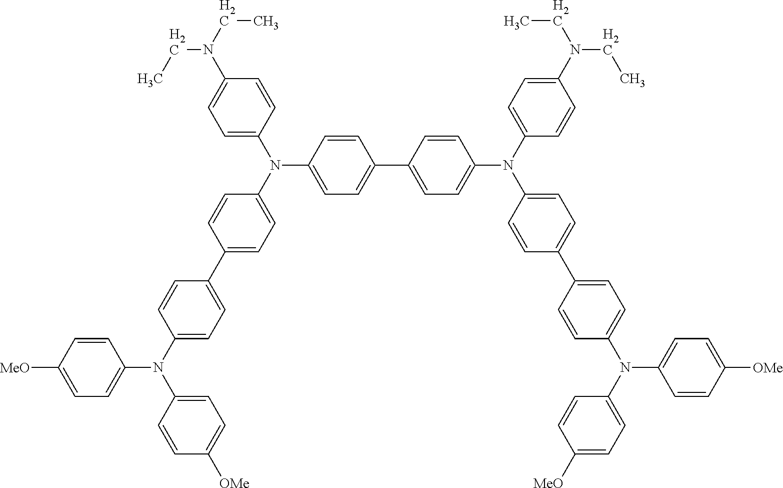

¹HNMR (400 MHz, THF-d₆): 7.36-7.42 (m, 12H), 6.99-7.07 (m, 20H), 6.90-6.92 (d, 4H), 6.81-6.84 (m, 8H), 6.66-6.69 (d, 4H), 3.74 (s, 12H), 3.36-3.38 (q, 8H), 1.41-1.17 (t, 12H)

### Example 16

### Compound ID522

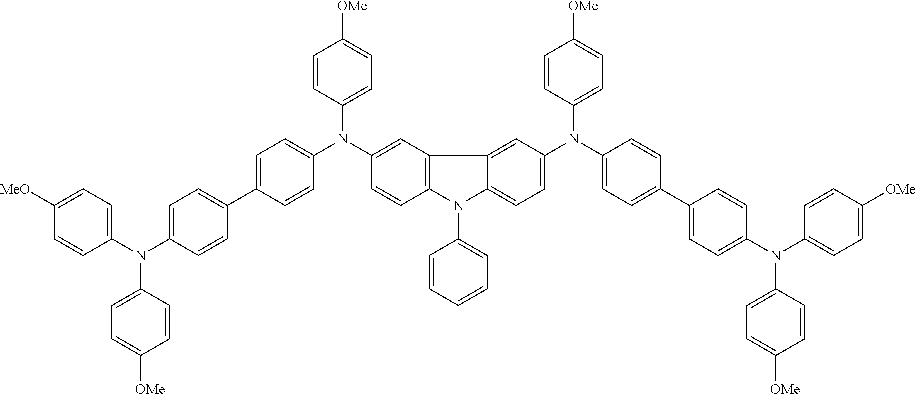

¹HNMR (400 MHz, DMSO-d₆): 7.65 (s, 2H), 7.52-7.56 (t, 2H), 7.44-7.47 (t, 1H), 7.37-7.39 (d, 2H), 7.20-7.22 (m, 10H), 7.05-7.08 (dd, 2H), 6.86-6.94 (m, 8H), 6.79-6.80-6.86 (m, 12H), 6.68-6.73, (dd, 8H), 6.60-6.62 (d, 4H), 3.68 (s, 12H), 3.62 (s, 6H)

### Example 17

### Compound ID523

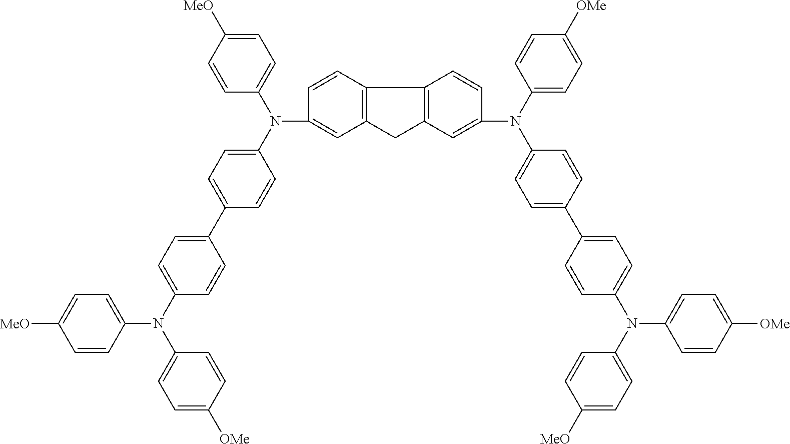

¹HNMR (400 MHz, THF-d₈): 7.54-7.56 (d, 2H), 7.35-7.40 (dd, 8H), 7.18 (s, 2H) 7.00-7.08 (m, 18H), 6.90-6.92 (d, 4H), 6.81-6.86 (m, 12H), 3.75 (s, 6H), 3.74 (s, 12H), 3.69 (s, 2H)

### Example 18

### Compound ID565

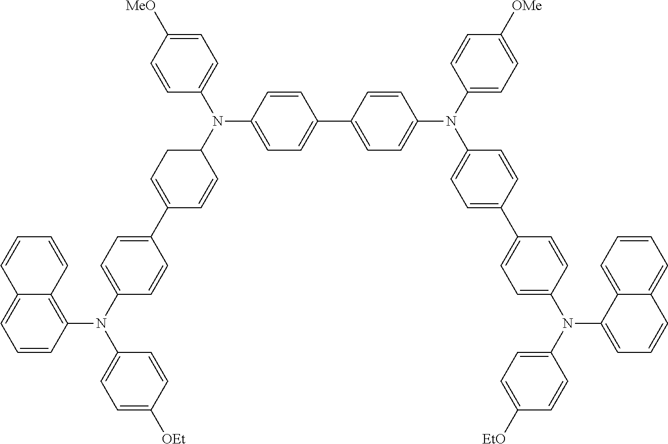

¹HNMR (400 MHz, THF-d8): 7.97-8.00 (d, 2H), 7.86-7.89 (d, 2H), 7.73-7.76 (d, 2H), 7.28-7.47 (m, 20H), 7.03-7.08 (m, 16H), 6.78-6.90 (m, 12H), 3.93-3.99 (q, 4H), 3.77 (s, 6H), 1.32-1.36 (s, 6H)

### Example 19

### Compound ID568

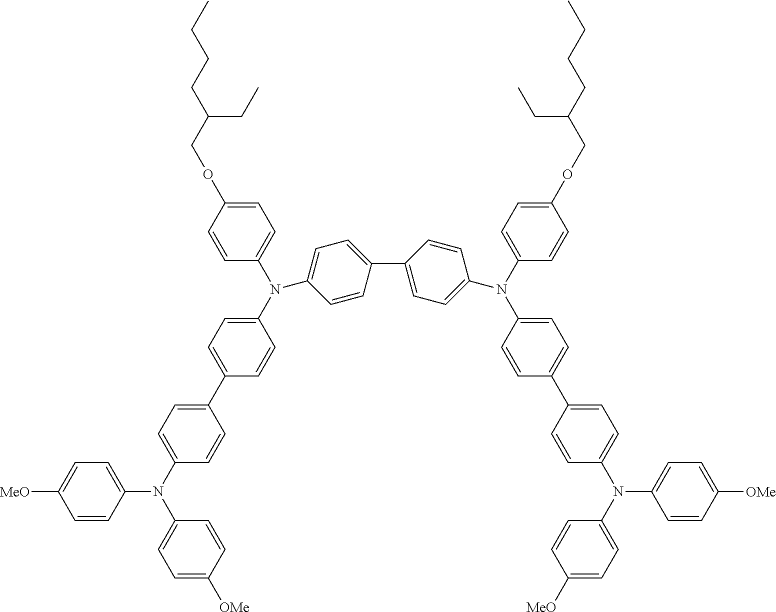

¹HNMR (400 MHz, DMSO-d6): 7.41-7.51 (m, 12H), 6.78-7.06 (m, 36H), 3.82-3.84 (d, 4H), 3.79 (s, 12H), 1.60-1.80 (m, 2H), 0.60-1.60 (m, 28H)

### Example 20

### Compound ID569

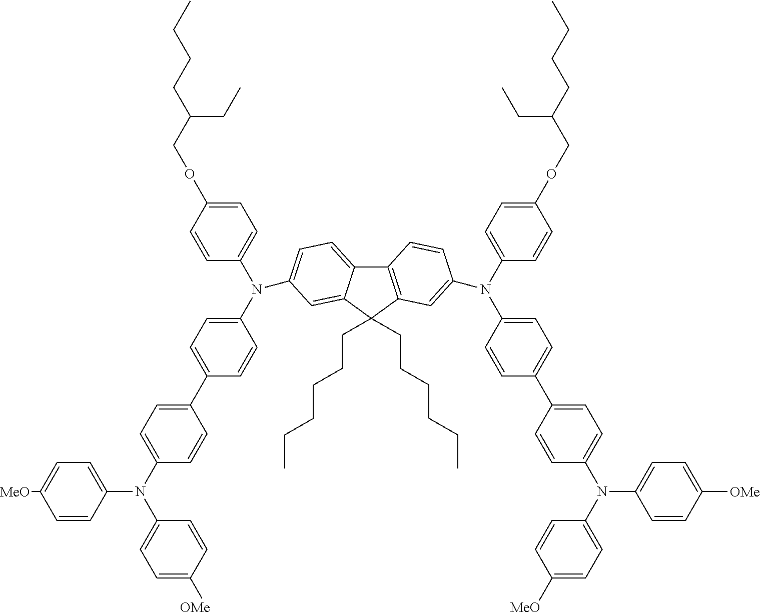

¹HNMR (400 MHz, DMSO-d6): 7.40-7.70 (m, 10H), 6.80-7.20 (m, 36H), 3.92-3.93 (d, 4H), 2.81 (s, 12H), 0.60-1.90 (m, 56H)

### Example 21

### Compound ID572

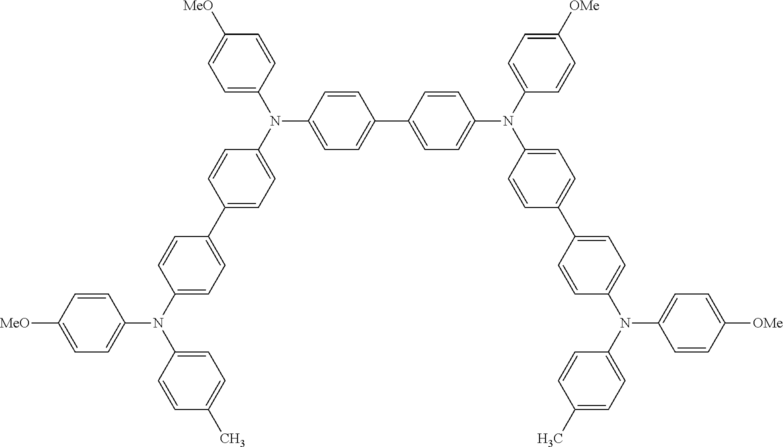

¹HNMR (400 MHz, THF-d8): 7.39-7.47 (m, 12H), 7.03-7.11 (m, 20H), 6.39-6.99 (m, 8H), 6.83-6.90 (m, 8H), 3.78 (s, 6H), 3.76 (s, 6H), 2.27 (s, 6H)

### Example 22

### Compound ID573

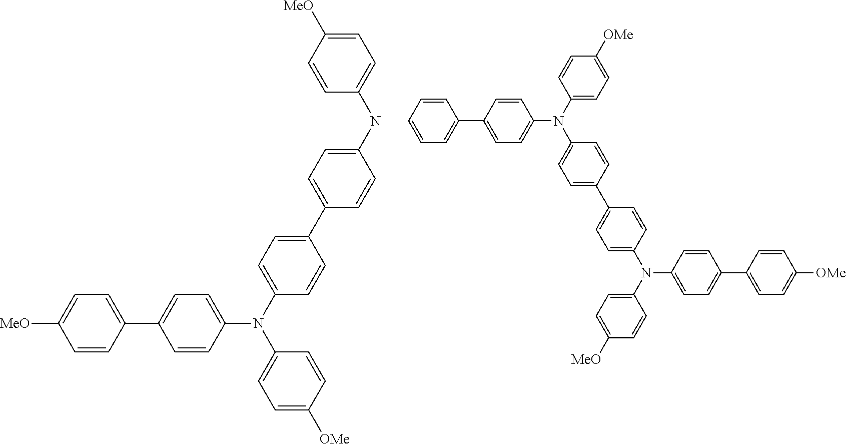

¹HNMR (400 MHz, THF-d8): 7.43-7.51 (m, 20H), 7.05-7.12 (m, 24H), 6.87-6.95 (m, 12H), 3.79 (s, 6H), 3.78 (s, 12H)

### Example 23

### Compound ID575

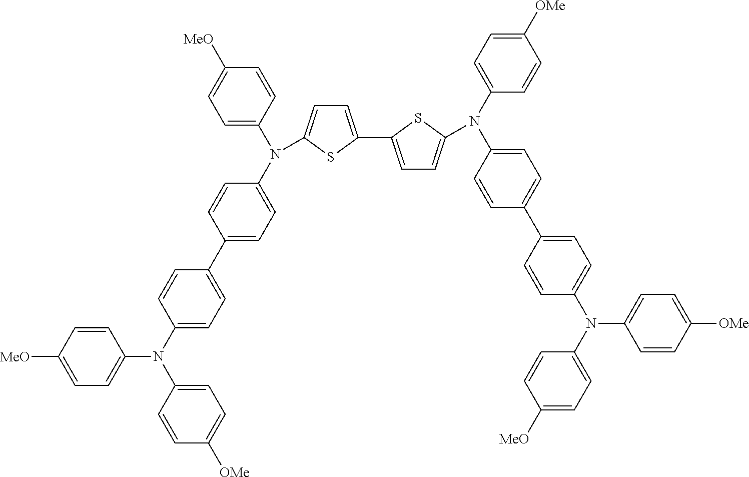

¹HNMR (400 MHz, DMSO-d6): 7.35-7.55 (m, 8H), 7.15-7.45 (m, 4H), 6.85-7.10 (m, 26H), 6.75-6.85 (d, 4H), 6.50-6.60 (d, 2H), 3.76 (s, 6H), 3.74 (s, 12H)

### Example 24

### Compound ID629

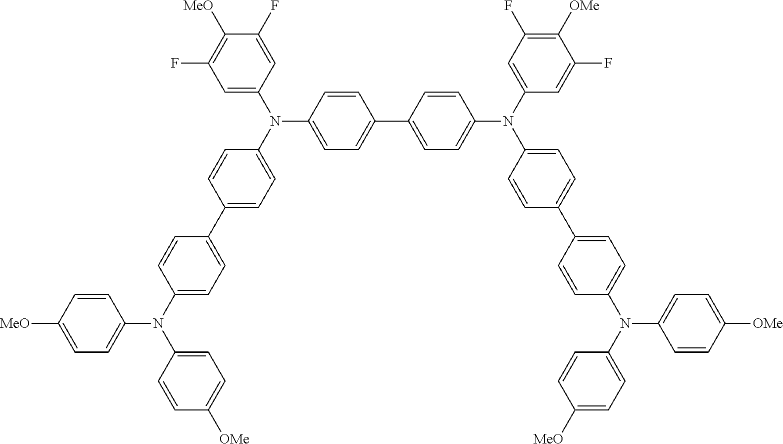

¹HNMR (400 MHz, THF-d₈): 7.50-7.56 (dd, 8H), 7.38-7.41 (dd, 4H), 7.12-7.16 (d, 8H) 7.02-7.04 (dd, 8H), 6.91-6.93 (d, 4H), 6.82-6.84 (dd, 8H), 6.65-6.68 (d, 4H) 3.87 (s, 6H), 3.74 (s, 12H)

### Example 25

### Compound ID631

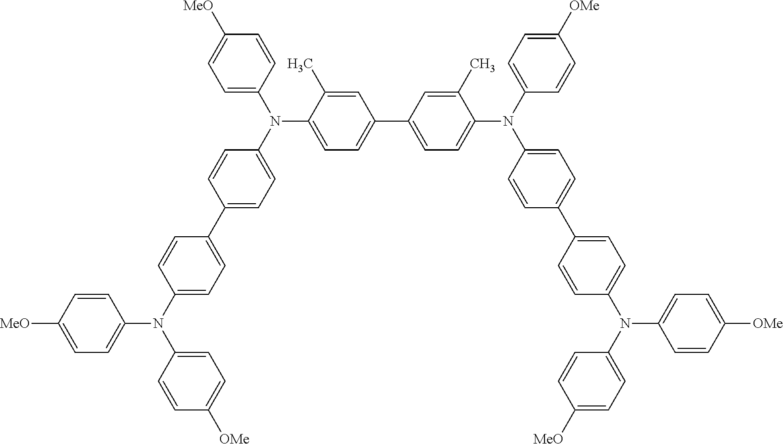

¹HNMR (400 MHz, THF-d₆): 7.52 (d, 2H), 7.43-7.47 (dd, 2H), 7.34-7.38 (m, 8H), 7.12-7.14 (d, 2H), 6.99-7.03 (m, 12H), 6.81-6.92 (m, 20H), 3.74 (s, 18H), 2.10 (s, 6H)

For spiro-MeOTAD and the compounds of examples 1 to 23, the glass transition temperature Tg (° C.) and the morphology M were determined in each case by means of DSC, the decomposition temperature Td by means of TGA (° C.), and the solubility L in chlorobenzene (mg/ml)—since this is one of the best known solvents for spiro-MeOTAD—at room temperature, and the data are compared in table 1 below. In the “M” column, the abbreviations here mean:
a: amorphous; only one glass transition at Tg could be detected here during the DSC measurement in the course of the first heating;
a*: the amorphous state was additionally confirmed by X-ray diffraction;
sc: semi-crystalline; the first heating during the DSC measurement resulted in crystallization after the glass transition at Tg;

TABLE 1

| Example | ID | Tg | M | Td | L |
|---|---|---|---|---|---|
| Spiro- | | 120 | sc | 440 | 200-230 |
| MeOTAD | | | | | |
| (comparative) | | | | | |
| 1 | 367 | 136 | a* | 450 | 700-1000 |
| 2 | 447 | 137 | a | 430 | 900-1800 |
| 3 | 453 | 156 | a | 440 | 450-700 |
| 4 | 320 | 96 | a* | 450 | 760-1000 |
| 5 | 321 | 143 | a* | 460 | 900-1150 |
| 6 | 366 | 127 | a* | 460 | >1100 |
| 7 | 368 | 154 | a | 460 | 500-1000 |
| 8 | 369 | 154 | a | 460 | 300-450 |
| 9 | 446 | 120 | a | 430 | 1250-2500 |
| 10 | 450 | 105 | a | 420 | 900-1800 |
| 11 | 452 | 123 | a | 450 | 650-1300 |
| 12 | 480 | 105 | a | 430 | >280 |
| 13 | 518 | 150 | a | 430 | 400-800 |
| 14 | 519 | 128 | a | 450 | 450-900 |
| 15 | 521 | 138 | a | 420 | 400-550 |
| 16 | 522 | 158 | a | 450 | 900-1400 |
| 17 | 523 | 141 | a | 450 | 1250-2500 |
| 18 | 565 | 143 | a | 450 | >900 |
| 19 | 568 | 65 | a | 360 | >650 |
| 20 | 569 | 47 | a | 440 | 400-800 |
| 21 | 572 | 138 | a | 450 | >900 |
| 22 | 573 | 148 | a | 460 | >1000 |
| 23 | 575 | 110 | a | 440 | >400 |
| 24 | 629 | 134 | a | 430 | >2000 |
| 25 | 631 | 134 | a | 440 | >1300 |

It can be inferred from table 1 that the compounds for use in accordance with the invention are all present in amorphous form. It can therefore be expected that they have a significantly lower tendency to crystallize and, with regard to a prolonged lifetime, bring about more advantageous properties of the DSCs produced therewith than DSCs based on the comparative compound spiro-MeOTAD. In addition, the compounds for use in accordance with the invention possess significantly better solubility than the comparative compound spiro-MeOTAD, which has a positive effect on the degree of pore filling in the production of the DSCs.

### Solubilities in Other Solvents:

Since chlorobenzene possesses low to moderate toxicity (LD50 of 2.9 g/kg according to Manfred Rossberg et al. “Chlorinated Hydrocarbons” in Ullmann's Encyclopedia of Industrial Chemistry Wiley-VCH, Weinheim, 2006) the compounds were also examined by way of example for their solubility in other solvents. As can be inferred from table 2, they have, in the predominant number of cases, increased solubility compared to spiro-MeOTAD (all data in mg/ml). In other words, the application of the hole conductors for use in accordance with the invention in high concentration is also possible from other solvents.

TABLE 2

| Solvent | | | | | |
|---|---|---|---|---|---|
| (Example) | | Toluene | Tetrahydrofuran | Ethyl acetate | Anisole |
| Spiro- | | 100 | 120-160 | <6 | 65-75 |
| MeO-TAD | | | | | |
| ID367 | (1) | 320-500 | 370-550 | 70-80 | 450-700 |
| ID447 | (2) | >520 | 750-1500 | >660 | n.d. |
| ID453 | (3) | 170-230 | 340-680 | 40 | n.d. |
| ID320 | (4) | 280 | >280 | 140 | n.d. |
| ID321 | (5) | >100 | >240 | 15 | n.d. |
| ID366 | (6) | >380 | >440 | >560 | n.d. |
| ID368 | (7) | >240 | 180-360 | >380 | n.d. |
| ID369 | (8) | 140-170 | 100-150 | 140-280 | n.d. |
| ID446 | (9) | >460 | 1000-2000 | 50-65 | n.d. |
| ID452 | (11) | >270 | 300-600 | n.d. | n.d. |
| ID518 | (13) | 160-240 | 550-1100 | >400 | n.d. |
| ID519 | (14) | 240-350 | >500 | >460 | n.d. |
| ID521 | (15) | 500-1000 | 440-580 | 90-110 | n.d. |
| ID522 | (16) | 490-980 | 400-600 | 600-1200 | n.d. |
| ID523 | (17) | 900-1800 | 400-550 | 240-480 | n.d. |
| ID565 | (18) | 400-800 | >850 | <40 | >1000 |
| ID572 | (21) | >500 | >800 | 60-100 | >400 |
| ID573 | (22) | 500-1000 | >580 | <20 | 550-1100 |
| ID629 | (24) | >1500 | >2000 | >1300 | >1500 |
| ID631 | (25) | 500-1000 | 1000-2000 | 700-1400 | 500-1000 |

“n.d.” means not determined
“>” in conjunction with a numerical value means that the solubility of the compound is greater than the numerical value reported.

B) Test of the Compounds for Use in Accordance with the Invention in DSCs:

The structure of a DSC generally comprises the following layers:
138 top contact (cathode)
124 hole-conducting material
123 hole-conducting material (in pores of the 120/122 layer)
122 sensitizing dye
120 n-semiconductive metal oxide
119 optional buffer layer
116 front contact (anode)
114 carrier

On the carrier 114 there is a layer 116 of a transparent conducting oxide (TCO), for example FTO (fluorine-doped tin oxide) or ITO (indium tin oxide). This TCO layer constitutes the front contact (anode).

An optional buffer layer 119 may be applied to the front contact, said buffer layer being intended to suppress or at least hinder the migration of the holes to the front electrode 116. The buffer layer 119 used is typically a single layer of a (preferably non-nanoporous) titanium dioxide and generally has a thickness between 10 nm and 500 nm. Such layers can be obtained, for example, by sputtering and/or spray pyrolysis.

The optional buffer layer 119 is followed by an about 1 μm- to 20 μm-thick layer 120 of a porous n-semiconductive metal oxide which is sensitized with a very thin, typically monomolecular layer 122 of a dye. The n-semiconductive metal oxide used is usually titanium dioxide, but other oxides are also conceivable.

The layer 120 of the n-semiconductive metal oxide sensitized with the dye (layer 122) is followed by a layer 123 of hole-conducting material. This material fills the pores of the layer 120/122 (metal oxide/dye) generally more or less completely, a maximum fill level being desirable. An interpenetrating layer/intimate permeation of hole-conducting material and n-semiconductive metal oxide/dye thus arises. In addition, the hole-conducting material on the metal oxide/dye layer forms a protruding layer 124 which is generally 10 nm to 500 nm thick, and one of its functions is to prevent electrons from passing out of the metal oxide into the cathode 138.

A counterelectrode 138 is applied as a top contact (cathode) to the layer 124.

In order to use the DSC, i.e. to be able to tap off a photovoltage and draw a photocurrent, a front contact (anode) 116 and the top contact (cathode) 138 are provided with appropriate conductive contacts, which, however, were not drawn in here for reasons of clarity.

### Dyes Used:

### D102:

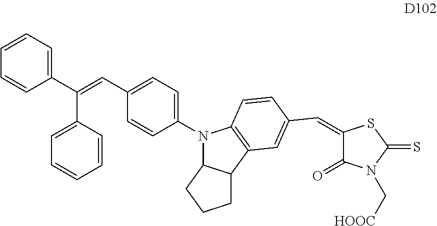

Commercially available from Mitsubishi.

### D205:

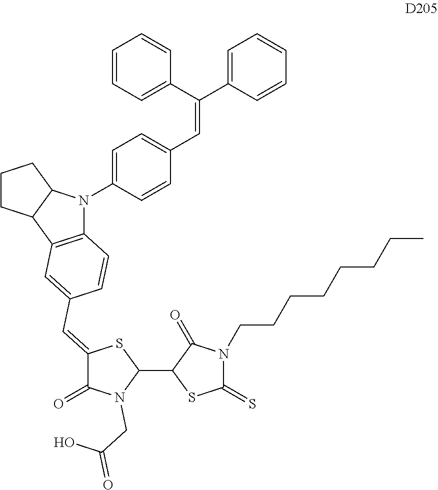

Synthesizable according to Seigo Ito et al., “High-conversion-efficiency organic dye-sensitized solar cells with a novel indoline dye”, Chem. Commun. 41, 5194-5196 (2008).

### Perylene 1:

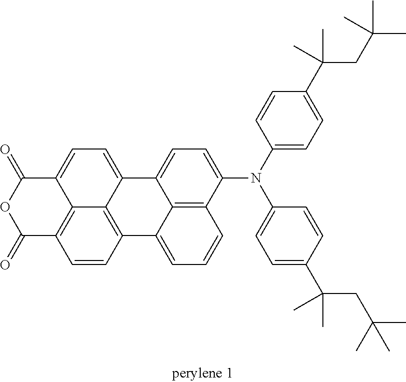

The synthesis was effected analogously to the synthesis of the compound I7 from example 7 on page 80 of WO 2007/054470 A1.

### Perylene 2:

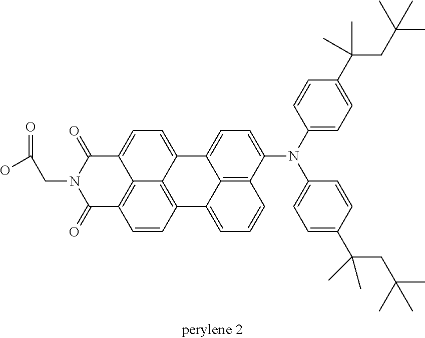

One equivalent of perylene 1 was reacted with 8 equivalents of glycine and one equivalent of anhydrous zinc acetate in N-methylpyrrolidone at 130° C. overnight. The product was purified using silica gel.

### Perylene 3:

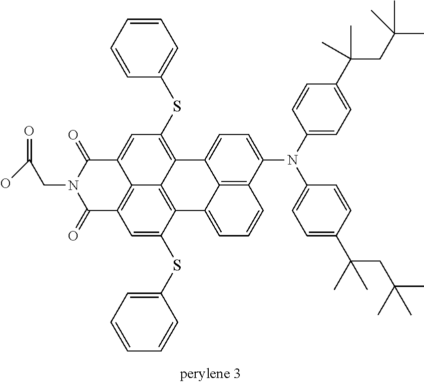

One equivalent of the compound I24 from example 24 on page 109 of WO 2007/054470 A1 was reacted with 8 equivalents of glycine and one equivalent of anhydrous zinc acetate in N-methylpyrrolidone at 130° C. overnight. The product was purified using silica gel.

The test DSCs were prepared as described below:

### DSC 1:

The base material used was glass plates coated with fluorine-doped tin oxide (FTO) of dimensions 25 mm×15 mm×3 mm (Nippon Sheet Glass), which were treated successively with glass cleaner (RBS 35), demineralized water and acetone, in each case in an ultrasound bath for 5 minutes, then boiled in isopropanol for 10 minutes and dried in a nitrogen stream.

To produce the solid TiO₂ buffer layer 119, a spray pyrolysis method as described in L. Kavan and M. Grätzel, Electrochim. Acta 40, 643 (1995) was used. Alternatively or additionally, it is, however, also possible to use other processes, for example sputtering processes (see P. Frach et al., Thin Solid Films 445 (2003) 251-258; D. Glöβ et al., Suf. coat. Technol. 200 (2005) 967-971).

A layer of an n-semiconductive metal oxide 120 was applied to the buffer layer 119. For this purpose, a TiO₂ paste (Dyesol, DSL 18NR-T) was applied by spinning with a spin coater at 4500 revolutions per minute and dried at 90° C. for 30 minutes. After heating to 450° C. for 45 minutes and sintering at 450° C. for 30 minutes, this resulted in a TiO₂ layer thickness of approximately 1.8 μm.

The intermediates thus produced were then treated with TiCl₄, as described by Grätzel, for example, in Grätzel M. et al., Adv. Mater. 2006, 18, 1202.

After removal from the sintering oven, the sample was cooled to 80° C. and immersed into a 0.5 mM solution of the dye D102 in 1:1 acetonitrile/t-BuOH for 12 hours. After removal from the solution, the sample was subsequently rinsed with the same solvent and dried in a nitrogen stream.

Next, the p-semiconductor ID367 was applied. For this purpose, a solution of 130 mM ID367, 12 mM LiN(SO₂CF₃)₂ (Aldrich), 47 mM 4-t-butylpyridine (Aldrich) in chlorobenzene was made up. 75 μl of this solution were applied to the sample and allowed to act for 60 seconds. Thereafter, the supernatant solution was spun off at 2000 revolutions per minute for 30 seconds and dried in ambient air for 3 hours.

The top contact (cathode) was applied by thermal metal evaporation under reduced pressure. For this purpose, the sample was provided with a mask in order to apply, by vapor deposition, 4 individual, separate rectangular top contacts with dimensions of in each case approx. 5 mm×4 mm to the active region, each of them connected to a contact area of about 3 mm×2 mm in size. The metal used was Ag, which was evaporated at a rate of 0.1 nm/s at a pressure of 5·10⁻⁵ mbar, so as to form a layer of thickness about 200 nm.

To determine the efficiency η, the particular current/voltage characteristic was measured with a Source Meter Model 2400 (Keithley Instruments Inc.) with irradiation with a xenon lamp (LOT-Oriel) as the solar simulator.

Current/voltage characteristics were measured at an illumination intensity of 10 mW/cm² (0.1 sun) and 100 mW/cm² (1 sun). The current density at 0.1 sun was multiplied by the factor of 10 in order to obtain the direct comparison to the measurement at 1 sun.

At 0.1 sun and 1 sun, the short-circuit current densities I_{SC} were 1.01 mA/cm² and 9.76 mA/cm² respectively, the terminal voltages V_{OC} with an open circuit 0.78 V and 0.86 V respectively, the fill factors (FF) 68% and 53% respectively, and the efficiencies 5.3% and 4.4% respectively.

### Comparative Example for DSC 1

As described in example DSC 1, a solid DSC was produced with the hole conductor spiro-MeO-TAD. For this purpose, a solution of 163 mM spiro-MeO-TAD, 15 mM LiN(SO₂CF₃)₂ (Aldrich), 60 mM 4-t-butylpyridine (Aldrich) in chlorobenzene was made up. At 0.1 sun and 1 sun, the short-circuit current densities I_{SC} were 1.10 mA/cm² and 10.60 mA/cm² respectively, the terminal voltages V_{OC} with an open circuit 0.74 V and 0.80 V respectively, the fill factors (FF) 69% and 47% respectively, and the efficiencies 5.6% and 4.0% respectively.

### DSC 2:

As described in the example DSC 1, a solid DSC was produced with the hole conductor ID447 and the dye D102 (hole conductor solution: 167 mM ID447, 15 mM LiN(SO₂CF₃)₂, 61 mM 4-t-butylpyridine in chlorobenzene).

At 0.1 sun and 1 sun, the short-circuit current densities I_{SC} were 0.91 mA/cm² and 6.95 mA/cm² respectively, the terminal voltages V_{OC} with an open circuit 0.72 V and 0.78 V respectively, the fill factors (FF) 56% and 33% respectively, and the efficiencies 3.8% and 1.8% respectively.

### DSC 3:

As described in the example DSC 1, a solid DSC was produced with the hole conductor ID453 and the dye D102 (hole conductor solution: 151 mM ID453, 14 mM LiN(SO₂CF₃)₂, 55 mM 4-t-butylpyridine in chlorobenzene).

At 0.1 sun and 1 sun, the short-circuit current densities I_{SC} were 0.87 mA/cm² and 7.75 mA/cm² respectively, the terminal voltages V_{OC} with an open circuit 0.84 V and 0.90 V respectively, the fill factors (FF) 61% and 34% respectively, and the efficiencies 4.5% and 2.3% respectively.

### DSC 4:

As described in the example DSC 1, a solid DSC was produced with the hole conductor ID522 and the dye D102 (hole conductor solution: 161 mM ID522, 15 mM LiN(SO₂CF₃)₂, 58 mM 4-t-butylpyridine in chlorobenzene). The thickness of the nanoporous TiO₂ layer this time was approx. 2.2 μm instead of approx. 1.8 μm.

At 0.1 sun and 1 sun, the short-circuit current densities I_{SC} were 0.83 mA/cm² and 8.77 mA/cm² respectively, the terminal voltages V_{OC} with an open circuit 0.76 V and 0.84 V respectively, the fill factors (FF) 69% and 45% respectively, and the efficiencies 4.4% and 3.3% respectively.

### Comparative Example for DSC 4

As described in the example DSC 4, a solid DSC was produced with the hole conductor spiro-MeO-TAD and the dye D102 (hole conductor solution: 163 mM spiro-MeO-TAD, 15 mM LiN(SO₂CF₃)₂ (Aldrich), 60 mM 4-t-butylpyridine (Aldrich) in chlorobenzene). The thickness of the nanoporous TiO₂ layer was approx. 2.2 μm as in the example DSC 4.

At 0.1 sun and 1 sun, the short-circuit current densities I_{SC} were 0.92 mA/cm² and 9.10 mA/cm² respectively, the terminal voltages V_{OC} with an open circuit 0.74 V and 0.82 V respectively, the fill factors (FF) 69% and 50% respectively, and the efficiencies 4.7% and 3.7% respectively.

### DSC 5:

As described in the example DSC 1, a solid DSC was produced with the hole conductor ID572 and the dye D102 (hole conductor solution: 178 mM ID572, 16 mM LiN(SO₂CF₃)₂, 65 mM 4-t-butylpyridine in chlorobenzene). The thickness of the nanoporous TiO₂ layer was approx. 1.8 μm as in example DSC 1.

At 0.1 sun and 1 sun, the short-circuit current densities I_{SC} were 0.91 mA/cm² and 8.52 mA/cm² respectively, the terminal voltages V_{OC} with an open circuit 0.84 V and 0.92 V respectively, the fill factors (FF) 58% and 40% respectively, and the efficiencies 4.5% and 3.1% respectively.

### DSC 6:

As described in the example DSC 1, a solid DSC was produced with the hole conductor ID367 and the dye D205 (hole conductor solution: 130 mM ID367, 12 mM LiN(SO₂CF₃)₂, 47 mM 4-t-butylpyridine in chlorobenzene). Dye bath: 0.5 mM solution of the dye D205 (as described in Schmidt-Mende et al., Adv. Mater. 2005, 17, 813) in 1:1 acetonitrile/t-BuOH.

At 0.1 sun and 1 sun, the short-circuit current densities I_{SC} were 0.97 mA/cm² and 8.92 mA/cm² respectively, the terminal voltages V_{OC} with an open circuit 0.80 V and 0.88 V respectively, the fill factors (FF) 70% and 46% respectively, and the efficiencies 5.4% and 3.7% respectively.

### Comparative Example for DSC6

As described in the example DSC6, a solid DSC was produced with the hole conductor spiro-MeO-TAD and the dye D205 (hole conductor solution: 123 mM spiro-MeO-TAD, 11 mM LiN(SO₂CF₃)₂, 45 mM 4-t-butylpyridine in chlorobenzene).

At 0.1 sun and 1 sun, the short-circuit current densities I_{SC} were 0.96 mA/cm² and 9.32 mA/cm² respectively, the terminal voltages V_{OC} with an open circuit 0.76 V and 0.86 V respectively, the fill factors (FF) 68% and 49% respectively, and the efficiencies 4.9% and 3.9% respectively.

### DSC 7:

As described in the example DSC 6, a solid DSC was produced with the hole conductor ID518 and the dye D205 (hole conductor solution: 202 mM ID518, 18 mM LiN(SO₂CF₃)₂, 74 mM 4-t-butylpyridine in chlorobenzene). The thickness of the nanoporous TiO₂ layer this time was 3.2 μm instead of approx. 1.8 μm.

At 0.1 sun and 1 sun, the short-circuit current densities I_{SC} were 0.81 mA/cm² and 8.57 mA/cm² respectively, the terminal voltages V_{OC} with an open circuit 0.74 V and 0.82 V respectively, the fill factors (FF) 63% and 33% respectively, and the efficiencies 3.8% and 2.3% respectively.

### Comparative Example for DSC 7

As described in the example DSC 7, a solid DSC was produced with the hole conductor spiro-MeO-TAD and the dye D205 (hole conductor solution: 204 mM spiro-MeO-TAD, 19 mM LiN(SO₂CF₃)₂, 74 mM 4-t-butylpyridine in chlorobenzene). The thickness of the nanoporous TiO₂ layer was, as in DSC 7, 3.2 μm instead of approx. 1.8 μm.

At 0.1 sun and 1 sun, the short-circuit current densities I_{SC} were 0.95 mA/cm² and 10.0 mA/cm² respectively, the terminal voltages V_{OC} with an open circuit 0.72 V and 0.78 V respectively, the fill factors (FF) 68% and 37% respectively, and the efficiencies 4.7% and 2.9% respectively.

### DSC 8:

As described in the example DSC 7, a solid DSC was produced with the hole conductor ID522 and the dye D205 (hole conductor solution: 201 mM ID522, 18 mM LiN(SO₂CF₃)₂, 73 mM 4-t-butylpyridine in chlorobenzene). The thickness of the nanoporous TiO₂ layer was, as in DSC 7, 3.2 μm instead of approx. 1.8 μm.

At 0.1 sun and 1 sun, the short-circuit current densities I_{SC} were 0.56 mA/cm² and 6.57 mA/cm² respectively, the terminal voltages V_{OC} with an open circuit 0.74 V and 0.84 V respectively, the fill factors (FF) 64% and 51% respectively, and the efficiencies 2.7% and 2.8% respectively.

### DSC 9:

As described in the example DSC 7, a solid DSC was produced with the hole conductor ID523 and the dye D205 (hole conductor solution: 214 mM ID523, 19 mM LiN(SO₂CF₃)₂, 78 mM 4-t-butylpyridine in chlorobenzene). The thickness of the nanoporous TiO₂ layer was, as in DSC 7, 3.2 μm instead of approx. 1.8 μm.

At 0.1 sun and 1 sun, the short-circuit current densities I_{SC} were 0.95 mA/cm² and 6.76 mA/cm² respectively, the terminal voltages V_{OC} with an open circuit 0.74 V and 0.80 V respectively, the fill factors (FF) 58% and 34% respectively, and the efficiencies 4.1% and 1.8% respectively.

### DSC 10:

As described in example DSC 1, a solid DSC was produced with the hole conductor ID367 and the dye perylene1 (dye bath: 0.5. mM solution of the dye perylene1 in dichloromethane). For this purpose, a solution of 130 mM ID367, 12 mM LiN(SO₂CF₃)₂ (Aldrich), 47 mM 4-t-butylpyridine (Aldrich) in chlorobenzene was made up.

At 0.1 sun and 1 sun the short-circuit current densities I_{SC} were 0.38 mA/cm² and 2.78 mA/cm² respectively, the terminal voltages V_{OC} with an open circuit 0.66 V and 0.74 V respectively, the fill factors (FF) 53% and 51% respectively, and the efficiencies 1.3% and 1.1% respectively.

### Comparative Example DSC 10

As described in example DSC 10, a solid DSC was produced with the hole conductor spiro-MeO-TAD and the dye perylene 1 (dye bath: 0.5 mM solution of the dye perylene 1 in dichloromethane). For this purpose, a solution of 123 mM spiro-MeO-TAD, 11 mM LiN(SO₂CF₃)₂ (Aldrich), 45 mM 4-t-butylpyridine (Aldrich) in chlorobenzene was made up.

At 0.1 sun and 1 sun, the short-circuit current densities I_{SC} were 0.37 mA/cm² and 2.58 mA/cm² respectively, the terminal voltages V_{OC} with an open circuit 0.60 V and 0.68 V respectively, the fill factors (FF) 55% and 54% respectively, and the efficiencies 1.2% and 0.9% respectively.

### DSC 11:

As described in example, DSC 1, a solid DSC was produced with the hole conductor ID367 and the dye perylene 2 (dye bath: 0.5 mM solution of the dye perylene 2 in dichloromethane). For this purpose, a solution of 130 mM ID367, 12 mM LiN(SO₂CF₃)₂ (Aldrich), 47 mM 4-t-butylpyridine (Aldrich) in chlorobenzene was made up.

At 0.1 sun and 1 sun, the short-circuit current densities I_{SC} were 0.42 mA/cm² and 4.39 mA/cm² respectively, the terminal voltages V_{OC} with an open circuit 0.78 V and 0.84 V respectively, the fill factors (FF) 68% and 54% respectively, and the efficiencies 2.3% and 2.0% respectively.

### Comparative Example DSC 11

As described in example DSC 10, a solid DSC was produced with the hole conductor spiro-MeO-TAD and the dye perylene 2 (dye bath: 0.5 mM solution of the dye perylene 2 in dichloromethane). For this purpose, a solution of 123 mM spiro-MeO-TAD, 11 mM LiN(SO₂CF₃)₂ (Aldrich), 45 mM 4-t-butylpyridine (Aldrich) in chlorobenzene was made up.

At 0.1 sun and 1 sun, the short-circuit current densities l_{SC} were 0.52 mA/cm² and 6.87 mA/cm² respectively, the terminal voltages V_{OC} with an open circuit 0.74 V and 0.76 V respectively, the fill factors (FF) 70% and 56% respectively, and the efficiencies 2.7% and 2.9% respectively.

### DSC 12:

As described in example DSC 1, a solid DSC was produced with the hole conductor ID523 and the dye perylene 3 (dye bath: 0.5 mM solution of the dye perylene 3 in dichloromethane). For this purpose, a solution of 214 mM ID523, 19 mM LiN(SO₂CF₃)₂ (Aldrich), 78 mM 4-t-butylpyridine (Aldrich) in chlorobenzene was made up. The thickness of the nanoporous TiO₂ layer was approx. 3.1 μm instead of approx. 1.8 μm.

At 0.1 sun and 1 sun, the short-circuit current densities I_{SC} were 0.21 mA/cm² and 3.40 mA/cm² respectively, the terminal voltages V_{OC} with an open circuit 0.64 V and 0.66 V respectively, the fill factors (FF) 64% and 59% respectively, and the efficiencies 0.9% and 1.3% respectively.

### Comparative Example DSC 12

As described in example DSC 12, a solid DSC was produced with the hole conductor spiro-MeO-TAD and the dye perylene 3 (dye bath: 0.5 mM solution of the dye perylene 3 in dichloromethane). For this purpose, a solution of 204 mM spiro-MeO-TAD, 19 mM LiN(SO₂CF₃)₂ (Aldrich), 74 mM 4-t-butylpyridine (Aldrich) in chlorobenzene was made up. The thickness of the nanoporous TiO₂ layer was approx. 3.1 μm instead of approx. 1.8 μm.

At 0.1 sun and 1 sun, the short-circuit current densities I_{SC} were 0.25 mA/cm² and 3.97 mA/cm² respectively, the terminal voltages V_{OC} with an open circuit 0.62 V and 0.66 V respectively, the fill factors (FF) 66% and 57% respectively, and the efficiencies 1.0% and 1.5% respectively.

1. A hole-conducting material comprising a compound of general formula I 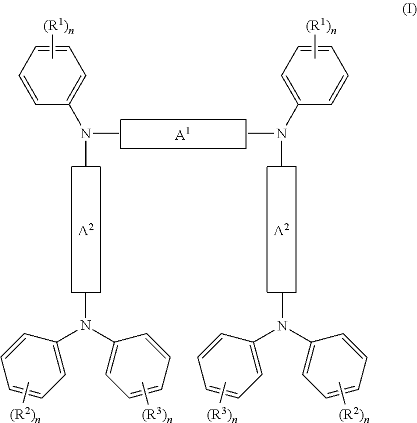
in which
A¹, A², A³ are each independently divalent organic units which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R¹, R², R³ are each independently R, OR, NR₂, A³-OR or A³-NR₂ substituents,
R is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl or NR′ radical,
R′ is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
and
n at each instance in formula I is independently 0, 1, 2 or 3,
wherein the sum of the individual values n is at least 2 and at least two of the R¹, R² and R³ radicals are OR and/or NR₂ substituents.
as hole conducting materials in organic solar cells.

2. The hole-conducting material comprising the compound according to claim 1, wherein at least two of the R¹, R² and R³ radicals are para-OR and/or -NR₂ substituents.

3. The hole-conducting material comprising the compound according to claim 1, wherein at least four of the R¹, R² and R³ radicals are para-OR and/or -NR₂ substituents.

4. The hole-conducting material comprising the compound according to claim 1, wherein all of the R¹, R² and R³ radicals are para-OR and/or —NR₂ substituents.

5. The hole-conducting material comprising the compound according to claim 1, wherein the organic A¹, A² and A³ units are selected from the group consisting of (CH₂)ₘ, C(R⁷)(R⁸), N(R⁹), 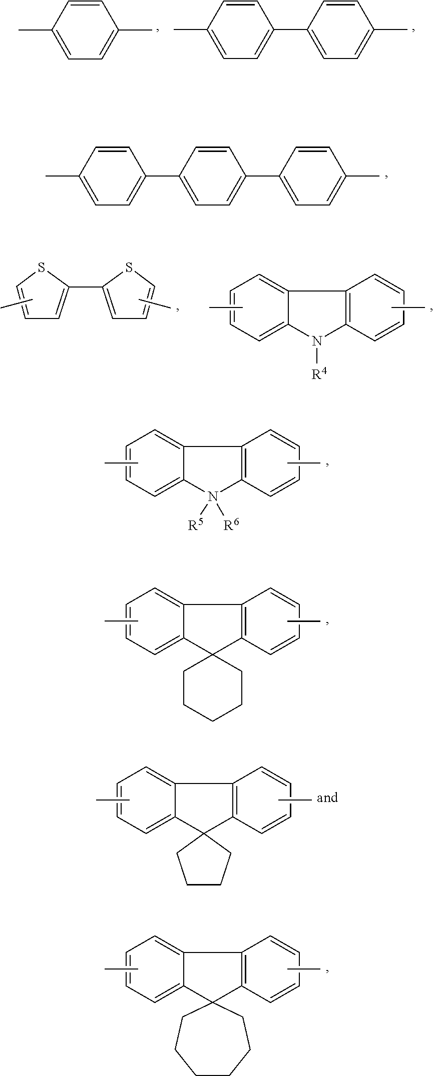
in which
m is an integer from 1 to 18,
R⁴, R⁹ are each alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R⁵, R⁶, R⁷, R⁸ are each independently hydrogen atoms or radicals as defined for R⁴ and R⁹,
and the aromatic and heteroaromatic rings of the units shown may have further substitution.

6. The hole-conducting material comprising the compound according to claim 1, wherein R in the R¹, R² and R³ radicals is independently C₁- to C₈-alkyl, cyclopentyl, cyclohexyl or aryl.

7. (canceled)

8. An organic solar cell comprising a compound of general formula I 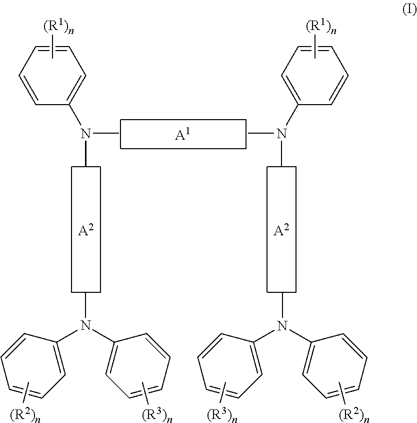
in which
A¹, A², A³ are each independently divalent organic units which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R¹, R², R³ are each independently R, OR, NR₂, A³-OR or A³-NR₂ substituents,
R is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl or NR′ radical,
R′ is alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
and
n at each instance in formula I is independently 0, 1, 2 or 3,
wherein the sum of the individual values n is at least 2 and at least two of the R¹, R² and R³ radicals are OR and/or NR₂ substituents.

9. The organic solar cell according to claim 8, wherein in the compound at least two of the R¹, R² and R³ radicals are para-OR and/or -NR₂ substituents.

10. The organic solar cell according to claim 8, wherein in the compound at least four of the R¹, R² and R³ radicals are para-OR and/or -NR₂ substituents.

11. The organic solar cell according to claim 8, wherein in the compound all of the R¹, R² and R³ radicals are para-OR and/or -NR₂ substituents.

12. The organic solar cell according to claim 8, wherein in the compound the organic A¹, A² and A³ units are selected from the group consisting of (CH₂)ₘ, C(R⁷)(R⁸), N(R⁹), 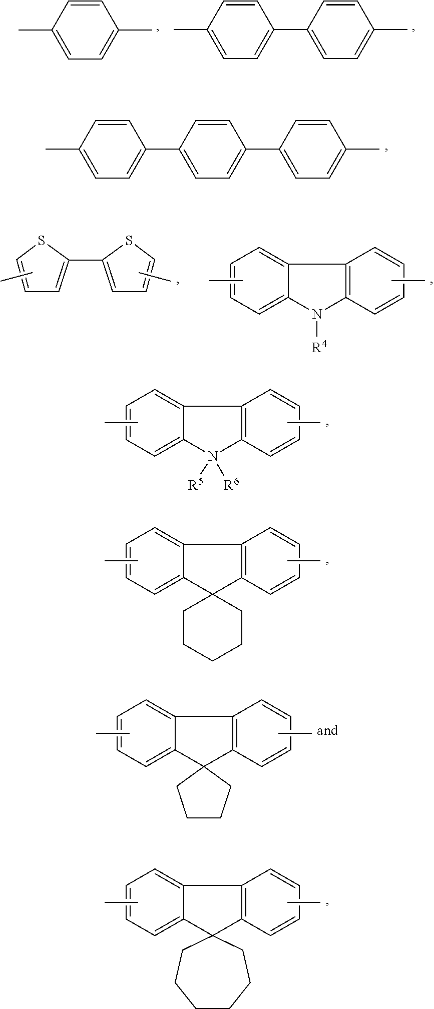
in which
m is an integer from 1 to 18,
R⁴, R⁹ are each alkyl, aryl or a monovalent organic radical which may comprise one, two or three optionally substituted aromatic or heteroaromatic groups, where, in the case of two or three aromatic or heteroaromatic groups, two of these groups in each case are joined to one another by a chemical bond and/or via a divalent alkyl radical,
R⁵, R⁶, R⁷, R⁸ are each independently hydrogen atoms or radicals as defined for R⁴ and R⁹,
and the aromatic and heteroaromatic rings of the units shown may have further substitution.

13. The organic solar cell according to claim 8, wherein in the compound R in the R¹, R² and R³ radicals is independently C₁- to C₈-alkyl, cyclopentyl, cyclohexyl or aryl.

14. The organic solar cell according to claim 8, wherein the solar cell comprises a plurality of layers, and one of said layers is a hole-conducting material layer comprising the compound.
